(19) United States
(12) Patent Application Publication
(10) Pub. No.: US 2021/0062273 A1
(43) Pub. Date: Mar. 4, 2021

(54) METHODS FOR RAPID DETECTION OF EGFR VARIANTS AND AMPLIFICATION

(71) Applicant: The Trustees of the University of Pennsylvania, Philadelphia, PA (US)

(72) Inventors: Deeksha Saxena, Ambler, PA (US); Jay F. Dorsey, Media, PA (US); Gary D. Kao, Wynnewood, PA (US); Donald O'Rourke, Wynnewood, PA (US)

(21) Appl. No.: 17/002,145

(22) Filed: Aug. 25, 2020

Related U.S. Application Data
(60) Provisional application No. 62/891,765, filed on Aug. 26, 2019.

(51) Int. Cl. C12Q 1/6886

(52) U.S. Cl. CPC C12Q 1/6886; C12Q 2600/158; C12Q 2600/156; C12Q 2600/112

(57) ABSTRACT

Provided herein are methods and compositions for detecting EGFR amplification and the presence of EGFR variants in a biological sample.

### STATEMENT OF FEDERALLY SPONSORED RESEARCH

This invention was made with government support under grant numbers 5R01CA201071 and R01CA181429 awarded by the National Institutes of Health. The government has certain rights in the invention.

### BACKGROUND OF THE INVENTION

Alterations in the Epidermal Growth Factor Receptor (EGFR) gene are a common finding in Glioblastoma (GBM).^{1,2} Variant III of EGFR occurs when an in-frame deletion of exon 2-7 leads to a deletion of 267 amino acids from the extracellular domain of the EGFR receptor³. EGFR receptors bearing variant III are unable to bind the canonical EGFR ligands, resulting in a constitutively active state, post-treatment tumor progression, radiation and chemotherapeutic resistance, and a poor prognosis.^{1,4} This novel domain stems from the union of normally distant parts of the protein, and creates an epitope that is not present in any normal tissues.⁵ Both amplified EGFR and EGFRvIII positive tumors have been linked to the invasive behavior of GBMs through several mechanisms.⁶

Despite available therapies, including surgical resection, radiotherapy, and chemotherapy, the median survival is less than two years for glioblastoma patients.⁷ Identification of patients that benefit from targeted therapeutic agents has been challenging. Agents targeting the EGFRvIII mutation have failed for multiple reasons including identifying a patient population that will benefit the most from these therapies.^{8,9} For instance, the novel peptide vaccine, Rindopepimut, which targets the unique epitope resulting from EGFRvIII, was well tolerated and produced an immune response.¹⁰ However, antigen escape variants were observed indicating that tumor heterogeneity may play an important role in guiding the appropriate selection of therapy following initial treatment.^{8,11} Another approach to targeting the EGFRvIII mutation is the use of Chimeric Antigen Receptor (CAR) T cells, which are genetically modified T cells engineered for enhanced reactivity against tumor antigens.¹² However, this approach has shown limited ability to address GBM recurrence.¹³

Current methods to detect EGFRvIII from tumor specimens include whole genome profiling using Next Generation Sequencing (NGS). Limitations to this approach include cost, availability of sufficient tissue, and the time required to execute the assay.¹⁴ For example, whole genome sequencing, Comparative Genomic Hybridization (CGH), and Single Nucleotide Polymorphism (SNP) arrays may be subject to high error rate when trying to identify specific subclonal populations.¹⁴ Serial monitoring of tumor genotype is also unfeasible with genome wide sequencing techniques.¹⁴ Alternatively, quantitative PCR may be used when small amounts of sample are available, but the low abundance of mutant DNA from human samples limits its quantitative impact.¹⁵ Detection of EGFRvIII through immunohistochemistry (IHC) has become more feasible with the advent EGFRvIII-specific antibodies. However, the intricacies of IHC protocols limit their diagnostic potential.

A need in the art exists for improved methods for detection of EGFR and EGFR variants.

### SUMMARY OF THE INVENTION

Provided herein are method for detection of EGFR and EGFR variants in a variety of biological samples. In certain embodiments, the method includes detecting epidermal growth factor receptor—variant III (EGFRvIII), the method comprising generating a cDNA sample from RNA present in a biological sample, contacting the cDNA sample with a forward primer that binds EGFR exon 1, a reverse primer that binds EGFR exon 8, and a minor groove binder (MGB) probe comprising an oligonucleotide having at least the sequence of: 5′ AAA GGT AAT TAT 3′ (SEQ ID NO: 3) that binds the junction of EGFR exons 1 and 8, and performing digital PCR in order to detect EGFRvIII. In certain embodiments, the forward primer comprises 5′ TCG GGC TCT GGA GGA AA 3′ (SEQ ID NO: 1) and/or the reverse primer comprises 5′ CTT CCT CCA TCT CAT AGC TGT C 3′ (SEQ ID NO: 2). In certain embodiments, EGFRvIII is detected with a limit of detection (LOD) of about 0.625×10⁻⁵ ng/ul and/or a limit of quantification (LOQ) of about 0.003%.

In yet a further embodiment, a method of detecting EGFR is provided, the method comprising generating a cDNA sample from RNA present in a biological sample, further comprising contacting the cDNA sample with a forward primer that binds EGFR exon 5, a reverse primer that binds EGFR exon 6, and a MGB probe comprising an oligonucleotide having at least the sequence of: 5′ AGG AGA ACT GCC 3′ (SEQ ID NO: 6) that binds the junction of EGFR exons 5 and 6, and performing digital PCR in order to detect wildtype EGFR. In certain embodiments, the forward primer that binds EGFR exon 5 comprises 5′ AAG TGT GAT CCA AGC TGT CC 3′ (SEQ ID NO: 4) and/or the reverse primer that binds EGFR exon 6 comprises 5′ TGC TGG GCA CAG ATG ATT T 3′(SEQ ID NO: 5).

In certain embodiments, the method comprises amplifying the cDNA generated from the RNA present in the biological sample. The biological sample can be selected from whole blood, PBMC, serum, a fresh, frozen, or preserved tumor sample, a tissue sample, CSF, a lymphatic fluid sample, a cell line, urine, and circulating tumor cells. In certain embodiments, the biological sample is tumor tissue. In certain embodiments, the the MGB probe is labeled with a dye selected from FAM, VIC, TET, and NED. In yet a further embodiment, digital PCR is performed using one cycle of 96° C. for 10 minutes, followed by 45 cycles of 54° C. for 2 minutes and 98° C. for 30 seconds and a final extension at 54° C. for 2 minutes.

In certain embodiments, a method for treating a cancer characterized by expression of epidermal growth factor receptor - variant III (EGFRvIII) is provided, the method comprising generating a cDNA sample from RNA present in the biological sample, contacting the cDNA sample with a forward primer that binds EGFR exon 1, a reverse primer that binds EGFR exon 8, and a MGB probe having an oligonucleotide comprising at least the sequence of: 5′ AAA GGT AAT TAT 3′ (SEQ ID NO: 3) that binds the junction of EGFR exon 1 and 8, and performing digital PCR in order to detect the EGFRvIII, wherein the presence of amplified sequences indicates a cancer associated with EGFRvIII, and treating the subject for the cancer. In certain embodiments, the sample is a brain tumor sample and the cancer is glioblastoma.

In certain embodiments, a method for treating a cancer characterized by amplified expression of EGFR is provided, the method comprising generating a cDNA sample from RNA present in the biological sample, contacting the cDNA sample with a forward primer that binds EGFR exon 5, a reverse primer that binds EGFR exon 5, and a MGB probe having an oligonucleotide comprising at least the sequence of 5′ AGG AGA ACT GCC 3′ (SEQ ID NO: 6) that binds the junction of EGFR exons 5 and 6, and performing digital PCR in order to detect the EGFR, wherein the presence of elevated expression of EGFR indicates a cancer associated with amplified EGFR expression, and treating the subject for the cancer. In certain embodiments, the sample is a brain tumor sample and the cancer is glioblastoma.

In certain embodiments, kits are provided for performing detection of EGFR and EGFR variants. In certain embodiments, the kit comprises a forward primer that binds EGFR exon 1, a reverse primer that binds EGFR exon 8, and a MGB probe comprising an oligonucleotide having at least the sequence of: 5′ AAA GGT AAT TAT 3′ (SEQ ID NO: 3) that binds the junction of EGFR exons 1 and 8. In certain embodiments, the kit comprises a forward primer that binds EGFR exon 5, a reverse primer that binds EGFR exon 6, and a MGB probe comprising an oligonucleotide having at least the sequence of: 5′ AGG AGA ACT GCC 3′ (SEQ ID NO: 6) that binds the junction of EGFR exons 5 and 6. In certain embodiments, the forward primer that binds EGFR exon 1 comprises 5′ TCG GGC TCT GGA GGA AA 3′ (SEQ ID NO: 1) and/or the reverse primer that binds EGFR exon 8 comprises 5′ CTT CCT CCA TCT CAT AGC TGT C 3′ (SEQ ID NO: 2). In certain embodiments, the forward primer that binds EGFR exon 5 comprises 5′ AAG TGT GAT CCA AGC TGT CC 3′ (SEQ ID NO: 4) and/or the reverse primer that binds EGFR exon 6 comprises 5′ TGC TGG GCA CAG ATG ATT T 3′(SEQ ID NO: 5). In certain embodiments, the kit includes additional reagents, including those suitable for processing a tissue sample, nucleic acid extraction from a biological sample, performing a reverse transcription reaction, and/or performing dPCR.

Other aspects and advantages of the invention will be readily apparent from the following detailed description of the invention.

### BRIEF DESCRIPTION OF THE DRAWINGS

FIG. 1 shows a schematic representation of a diagnostic digital PCR assay. A tumor specimen is collected at the time of resection. This specimen is either snap frozen in liquid nitrogen, stored at −20° C. for immediate processing, or stored in PreservCyt® media. RNA is extracted from the sample mentioned as well as from FFPE slides and then undergoes first strand cDNA synthesis followed by magnetic bead separation. cDNA is analyzed via a digital PCR platform using unique primer-probe combinations to detect EGFR WT and EGFRvIII. The highly multiplex nature of digital PCR allows for quantification of EGFR WT and EGFRvIII copy numbers. Total assay time is less than twenty-four hours.

FIG. 2A-FIG. 2F show a schematic representation of the location of MGB probes and primers on EGFR and EGFRvIII. (FIG. 2A) The EGFR WT FAM labeled MGB probe recognizes the junction of exon 5 and exon 6. Forward and reverse primers flank either side of the probe. (FIG. 2B) The EGFRvIII MGB probe is labeled with either FAM or VIC and recognizes the junction of exon 1 and exon 8. Forward and reverse primers pair with nucleotide sequences on either side of the MGB probe. (FIG. 2C) MGB probe and primer pair nucleotide sequences for EGFR WT and EGFRvIII. (FIG. 2D) Digital PCR reaction conditions were optimized using EGFR WT primers and probe and a dilution series of EGFR WT plasmid DNA concentrations ranging from 0.625E-5 ng/μL to 5E-5 ng/μL. Our assay was able to produce a concentration-dependent increase in the copies per microliter of EGFR WT. The lowest quantity of EGFR WT DNA detected using our dPCR assay was 0.625E-5 ng/μL. (FIG. 2E) Different concentrations of EGFRvIII plasmid DNA ranging from 0.625E-5 ng/μL to 5E-5 ng/μL were assayed using our digital PCR assay and EGFRvIII primers and probe producing a concentration-dependent increase in the copies per microliter of EGFRvIII. The lowest quantity of EGFRvIII DNA detected was 0.625E⁻⁵ ng/μL (FIG. 2F) EGFRvIII plasmid DNA was assayed at concentrations ranging from 0.23 pg to 1.9 pg in a background of 6000 pg EGFR WT plasmid DNA. The Limit of Quantification of our EGFRvIII assay is 0.23 pg of EGFRvIII DNA with sensitivity as low as 0.003%.

FIG. 3A-FIG. 3F show the determination of EGFR WT and EGFRvIII expression on U87 cell lines. (FIG. 3A) Western blot analysis confirming that U87 EGFRvIII over-express the variant III mutation of EGFR. (3-actin serves as a loading control. (FIG. 3B) Images taken at 63× magnification (scale bar=20 microns) demonstrate that the mutant isoform of EGFR is present in the U87 EGFRvIII cell line. DAPI counter-stain highlights the cytoplasmic localization of EGFRvIII. (FIG. 3C) Conventional PCR reaction confirms the presence of variant III mutation in U87 EGFRvIII cells when compared to U87 EGFR WT cells. (FIG. 3D) Representative images of U87 EGFR WT and U87 EGFRvIII cells taken at 10X magnification before and after Kuiqpick harvesting of cells (scale bar=100 microns). U87 EGFR WT and vIII were labeled with a phycoerythrin tracer, and were mixed with unlabeled U87 EGFRvIII and U87 EGFR WT that had been infected with our telomerase based adenovirus probe to label all cells with GFP. Mixed cells were then plated and imaged twenty-four hours later. Arrow points indicate cells that were selected via kuiqpick. (FIG. 3E) cDNA extracted from kuiqpicked U87 EGFR WT and vIII cells^{(1, 5, 10, 50)} were amplified using EGFR open reading frame primers to confirm pure populations. β-actin primers were used to confirm a successful PCR reaction. Amplified PCR product using EGFR ORF primers were subjected to TOPO TA cloning and then sequenced. The EGFR transcript variant 1 (accession NM_005228) sequence was acquired from NCBI's RefSeq database. Sequence readings were trimmed and aligned to the reference using Geneious version 7.1.8. (FIG. 3F) Upper panel, Representative Sanger sequencing of one clone of EGFR WT from a single cell of U87 EGFR WT emphasizing the exon 1-2 continuity of the EGFR WT sequence. Lower panel, Representative Sanger sequencing of one clone of EGFRvIII obtained from a single cell of U87 EGFRvIII, showing the unique fusion junction between exons 1 and 8.

FIG. 4A-FIG. 4D show the lower limit of detection of a EGFRvIII digital PCR assay. (FIG. 4A) The digital PCR assay containing probe and primer pairs against EGFR wildtype was able to detect wildtype copies in U87 WT as well as U87 vIII cDNA at concentrations as low as 1.25E-3 ng/μL.(FIG. 4B) Changing the probe and primer pairs allowed for detection of EGFRvIII in U87 vIII cDNA at concentrations as low as 1.25E-3 ng/μL. EGFRvIII was not detected in U87 WT cDNA. Representative scatter plots of vIII expression (dark gray) of U87 EGFR WT(FIG. 4C) and U87 EGFRvIII (FIG. 4D) cDNA at a concentration of 2.5E-3 ng/μL, respectively. Non-amplified wells are denoted in (light gray).

FIG. 5A-FIG. 5F show detection of EGFRvIII in patient-derived glioma neurospheres. (FIG. 5A) cDNA extracted from various patient derived glioma neurosphere cell lines were amplified using EGFR ORF primers (upper panel), β-Actin primers (lower panel). (FIG. 5B) Western blot analysis confirms the expression of EGFR WT and EGFRvIII in various patient-derived glioma neurospheres. (FIG. 5C) Graphical representation of copies per microliter generated when dPCR assay was run on cDNA extracted from various patient derived glioma neurospheres-EGFR WT (light bars) and EGFRvIII (dark bars). NS039 and HK296 both have abundant copies of vIII, whereas HK301 and HK296 have an abundance of EGFR WT. (FIG. 5D) Copies per microliter of EGFR WT and EGFRvIII generated when our dPCR assay was run on cDNA extracted from NS039 and T4213 cells spiked into mouse blood. These results emphasize that additional cell types do not affect the outcome of our assay. (FIG. 5E and FIG. 5F) Representative scatter plots of vIII expression (dark gray) from NS039 cells (FIG. 5E) and T4213 (FIG. 5E) spiked into mouse blood following our digital PCR assay. Non-amplified wells are denoted in (light gray).

FIG. 6A-FIG. 6F shows EGFRvIII expression in orthotopic glioma xenografts. (FIG. 6A) Serial bioluminescent images over a period of a few weeks from mice that underwent orthotopic implantation of U87 EGFRvIII cells indicating tumor growth. (FIG. 6B) A similar set of bioluminescent images over a period of a few weeks from mice that underwent orthotopic implantation of EGFRvIII-negative T4213 cells confirming tumor growth. (FIG. 6C) Tumor tissue was collected from mice four to six weeks following orthotopic implantation of U87 vIII or T4213 cells. Digital PCR analysis identified EGFRvIII in U87 orthotopic tumors, but not in T4213 tumors. (FIG. 6D and FIG. 6E) Representative scatter plots of vIII copies from U87 vIII and T4213 orthotopic tumors. Non-amplified wells are denoted in light gray. (FIG. 6F) Comparison of vIII and WT expression across different tumor types including T4213 orthotopic, NS039 flank, and U87 vIII orthotopic tumors.

FIG. 7A-FIG. 7D show EGFRvIII expression in patient tumor samples. (FIG. 7A) Representative scatter plots of EGFRvIII (dark gray) and EGFR WT (dark gray) copies from patient tumor sample Frozen 1 (Fr 1) received from the University of Pennsylvania tumor bank. (FIG. 7B) Representative scatter plots of EGFRvIII (dark gray) and EGFR WT (dark gray) copies from patient sample Frozen 2 (Fr 2). Digital PCR analysis reveals that in both patient samples vIII is detected and WT is over-amplified. (FIG. 7C) and (FIG. 7D) Representative scatter plots of from two EGFRvIII negative patient samples (FIG. 7C-FFPE 14 sample, and FIG. 7D—Fresh Primary tumor F1A). Lighter gray dots represent non-amplified wells. No expression of EGFRvIII is observed in these samples.

FIG. 8 depicts a table showing clinical diagnosis and demographic characteristics of patient samples. dPCR values represents summary of expression of EGFRvIII and EGFR WT.

FIG. 9 depicts a table showing comparison of storage of twenty-one patient tumor samples in −20° C. vs PreservCyt® in RNA extraction and dPCR analysis. PreservCyt® did not appreciably impact RNA extraction or the results of dPCR analyses.

FIG. 10 shows EGFRvIII and EGFR WT expression on from cell free RNA (cfRNA). Cell-free RNA was extracted from GSC (NS039, HK248, HK296, T4213), U87 WT and, U87 vIII. In a separate experiment cell-free RNA from U87 vIII growth media was spiked into (mouse or human) whole blood). Extracted RNA was converted to cDNA and assayed for vIII and WT using our digital PCR assay. Similar patterns of EGFR WT and vIII expression were observed following digital PCR analysis of cell free RNA as was observed in cDNA from the parental cell lines.

FIG. 11A-FIG. 11G show detection of missense EGFR mutations R108K and A289V in patient tumor samples and patient-derived organoids.

### DETAILED DESCRIPTION OF THE INVENTION

Provided herein are methods, both quantitative and qualitative, and compositions for detecting EGFR and EGFR variants in a biological sample. In certain embodiments, the methods include diagnosing and treating a cancer based on the presence of or levels of expression of wildtype EGFR and/or an EGFR variant in a biological sample. In particular, methods are provided for diagnosing or detecting EGFRvIII in small amounts of spatial heterogeneous tissue in a rapid fashion. Improved detection of EGFR and EGFRvIII facilitates early intervention with a cancer treatment and monitoring of a patient's response to treatment.

The epidermal growth factor receptor (EGFR), also known as HER1 and ERBB1, is a pivotal regulator of normal cellular growth in tissues of epithelial origin. Dysregulated EGFR signaling (resulting from mechanisms such as cell-surface overexpression, autocrine activation and EGFR gene mutation) contributes to the formation of many epithelial malignancies in humans. Where dysregulation is the result of cell-surface EGFR overexpression, there is often associated gene amplification and/or mutation. Although several EGFR mutations have been described, the most common extracellular mutation is EGFRvIII (also known as de2-7EGFR and DEGFR). EGFRvIII is a tumor-specific mutation that results from in-frame deletion of 801 base pairs spanning exons 2-7 of the coding sequence. This deletion removes 267 amino acids from the extracellular domain, creating a junction site between exons 1 and 8 and a new glycine residue. EGFRvIII has a molecular mass of approximately 145 kDa (see, e.g., An et. Oncogene. 2018 March; 37(12), which is incorporated herein by reference). Other mutations may be detected as described herein including, e.g., missense EGFR mutations R108K and/or A289V (amino acid numbering as provided in SEQ ID NO: 15). Sequences for human EGFRvIII are available, for example, from the NCBI database as accession NM_001346941.2.

Provided herein are methods for detection of EGFR and/or EGFRvIII in a biological sample from a patient. In certain embodiments, the patient that has been diagnosed with or is suspected of having a high-grade brain tumor such as glioblastoma multiforme (GBM), the most common and lethal malignant neoplasm of the brain in adults. EGFRvIII is known to be associated with aggressive pathological features in GBM, including enhanced tumorigenicity, invasion, and therapeutic resistance. Thus, in certain embodiments, the detection of EGFR amplification and/or the EGFRvIII mutation in a biological sample is associated with a poor prognosis in a subject with GBM.

As used herein, the term “biological sample” refers to any cell, biological fluid, or tissue that comprises nucleic acids. Suitable samples for use in this invention may include, without limitation, whole blood, a blood fraction (e.g. peripheral blood mononuclear cells (PBMC)), leukocytes, fibroblasts, serum, urine, lymphatic fluid, plasma, saliva, bone marrow, cerebrospinal fluid, ascitic fluid, amniotic fluid, skin cells, cerebrospinal fluid, cervical secretions, vaginal secretions, endometrial secretions, gastrointestinal secretions, bronchial secretions, cell line, tissue sample, or secretions from the breast. Such samples may further be diluted with saline, buffer or a physiologically acceptable diluent. Alternatively, such samples are concentrated by conventional means. Biological samples may also include sections of tissues such as biopsy and autopsy samples, FFPE samples, and frozen sections taken for histological purposes. In certain embodiments, the biological sample was collected and/or stored in PreservCyt® medium (ThinPrep Pap Test; Cytyc Corporation, Boxborough, Mass.) prior to isolation of RNA. In certain embodiments, the biological sample is a cell-free nucleic acid sample having nucleic acid released by cells (for example, culture supernatant, CSF fluid, plasma, or an exosome preparation). In certain embodiments, the biological sample is a tumor extract or circulating tumor cells. In yet a further embodiment, the biological sample is an organoid.

A biological sample may be provided by removing a sample from a subject but can also be accomplished by using a previously isolated sample or cells (e.g., isolated by another person, at another time, and/or for another purpose). Archival tissues, such as those having treatment or outcome history, may also be used. Biological samples also include explants and primary and/or transformed cell cultures derived from animal or human tissues. Methods for detection of EGFR variants in samples, particularly those of diminished or poor quality are provided, for example, by Kim et al. Droplet digital PCR-based EGFR mutation detection with an internal quality control index to determine the quality of DNA, Sci Rep. 2018 Jan. 11; 8(1):543, which is incorporated by reference herein.

As used herein, the term “subject” means a mammalian animal, including a human, a veterinary or farm animal, a domestic animal or pet, and animals normally used for clinical research. In one embodiment, the subject of these methods and compositions is a human. As used herein, the term “subject” is used interchangeably with “patient”. In certain, embodiments the subject has been diagnosed or is suspected of having glioblastoma multiforme (GBM).

As used herein, “a healthy control” refers to a subject or a biological sample therefrom, wherein the subject does not amplification of EGFR or an EGFR variant of interest. The healthy control can be from one subject. In another embodiment, the healthy control is a pool of multiple subjects.

The term “cancer” is meant to include all types of cancerous growths or oncogenic processes, metastatic tissues or malignantly transformed cells, tissues, or organs, irrespective of histopathologic type or stage of invasiveness. Examples of cancers include, but are not limited, to solid tumors and leukemias, including: apudoma, choristoma, branchioma, malignant carcinoid syndrome, carcinoid heart disease, carcinoma (e.g., Walker, basal cell, basosquamous, Brown-Pearce, ductal, Ehrlich tumor, non-small cell lung (e.g., lung squamous cell carcinoma, lung adenocarcinoma and lung undifferentiated large cell carcinoma), oat cell, papillary, bronchiolar, bronchogenic, squamous cell, and transitional cell), histiocytic disorders, leukemia (e.g., B cell, mixed cell, null cell, T cell, T-cell chronic, HTLV-II-associated, lymphocytic acute, lymphocytic chronic, mast cell, and myeloid), histiocytosis malignant, Hodgkin disease, immunoproliferative small, non-Hodgkin lymphoma, plasmacytoma, reticuloendotheliosis, melanoma, chondroblastoma, chondroma, chondrosarcoma, fibroma, fibrosarcoma, giant cell tumors, histiocytoma, lipoma, liposarcoma, mesothelioma, myxoma, myxosarcoma, osteoma, osteosarcoma, Ewing sarcoma, synovioma, adenofibroma, adenolymphoma, carcinosarcoma, chordoma, craniopharyngioma, dysgerminoma, hamartoma, mesenchymoma, mesonephroma, myosarcoma, ameloblastoma, cementoma, odontoma, teratoma, thymoma, trophoblastic tumor, adeno-carcinoma, adenoma, cholangioma, cholesteatoma, cylindroma, cystadenocarcinoma, cystadenoma, granulosa cell tumor, gynandroblastoma, hepatoma, hidradenoma, islet cell tumor, Leydig cell tumor, papilloma, Sertoli cell tumor, theca cell tumor, leiomyoma, leiomyosarcoma, myoblastoma, myosarcoma, rhabdomyoma, rhabdomyosarcoma, ependymoma, ganglioneuroma, glioma, medulloblastoma, meningioma, neurilemmoma, neuroblastoma, neuroepithelioma, neurofibroma, neuroma, paraganglioma, paraganglioma nonchromaffin, angiokeratoma, angiolymphoid hyperplasia with eosinophilia, angioma sclerosing, angiomatosis, glomangioma, hemangioendothelioma, hemangioma, hemangiopericytoma, hemangiosarcoma, lymphangioma, lymphangiomyoma, lymphangiosarcoma, pinealoma, carcinosarcoma, chondrosarcoma, cystosarcoma, phyllodes, fibrosarcoma, hemangiosarcoma, leimyosarcoma, leukosarcoma, liposarcoma, lymphangiosarcoma, myosarcoma, myxosarcoma, ovarian carcinoma, rhabdomyosarcoma, sarcoma (e.g., Ewing, experimental, Kaposi, and mast cell), neurofibromatosis, and cervical dysplasia, and other conditions in which cells have become immortalized or transformed. In certain embodiments, the cancer is glioblastoma multiforme (GBM).

Provided herein are methods of detecting or measuring levels of expression of epidermal growth factor receptor—variants R108K and/or A289V in a biological sample using digital PCR (dPCR). In certain embodiments, the method includes contacting cDNA generated from the RNA present in a biological sample with a forward primer that binds EGFR exon 1, a reverse primer that binds EGFR exon 1, and a minor groove binder (MGB) probe. In certain embodiments, the method includes contacting cDNA generated from the RNA present in a biological sample with a forward primer that binds EGFR exon 2, a reverse primer that binds EGFR exon 2, and a minor groove binder (MGB) probe. In certain embodiments, the method includes contacting cDNA generated from the RNA present in a biological sample with a forward primer that binds EGFR exon 1, a reverse primer that binds EGFR exon 2, and a minor groove binder (MGB) probe. cDNA generated from RNA present in a biological sample can be reversed transcribed to generate cDNA using routine methods and protocols known to one of skill in the art. Further, in certain embodiments, the cDNA generated from RNA present in the biological sample is further amplified prior to performing dPCR. In certain embodiments, the MGB probe used for methods of detecting or measuring EGFR variants R108K and/or A289V includes a fluorescent label (or dye), such as, for example, FAM, VIC, TET, and NED.

Provided herein are methods of detecting or measuring levels of expression of epidermal growth factor receptor - variant III (EGFRvIII) in a biological sample using digital PCR (dPCR). The methods allow for detection of EGFRvIII in patient-derived tissue specimens with high levels of sensitivity and allow for rapid and accurate characterization of the EGFRvIII variant in brain tumors and other solid tumors. For example, the methods provided herein enable quantification of EGFRvIII at the level of one picogram in a background of six thousand picograms of EGFR wildtype. Further, the methods allow for detection of EGFRvIII in patient-derived tissue specimens, including brain tumors and other solid tumors, where next generation sequencing (NGS) is unable to detect the presence of the mutation. In certain embodiments, the methods allow for detection of EGFRvIII in a biological sample within a day of resection. Accordingly, the methods provided are particularly useful for detection of EGFR variants, such as EGFRvIII,without the need to sequence the whole genome of a subject.

In certain embodiments, the method includes contacting cDNA generated from the RNA present in a biological sample with a forward primer that binds EGFR exon 1, a reverse primer that binds EGFR exon 8, and a minor groove binder (MGB) probe having a oligonucleotide that binds the junction of EGFR exons 1 and 8. RNA present in a biological sample can be reversed transcribed to generate cDNA using routine methods and protocols known to one of skill in the art. Further, in certain embodiments, the cDNA generated from RNA present in the biological sample is further amplified prior to performing dPCR. In certain embodiments, the MGB probe used for methods of detecting or measuring EGFRvIII includes a fluorescent label (or dye), such as, for example, FAM, VIC, TET, and NED.

In certain embodiments, the forward primer that binds EGFR exon 1 includes the sequence 5′ TCG GGC TCT GGA GGA AA 3′ (SEQ ID NO: 1). In yet further embodiments, the forward primer that binds EGFR exon 1 is 5′ TCG GGC TCT GGA GGA AA 3′. In yet further embodiments, the forward primer that binds EGFR exon 1 has a sequence that is substantially identical to the sequence 5′ TCG GGC TCT GGA GGA AA 3′.

In certain embodiments, the reverse primer that binds EGFR exon 8 includes the sequence 5′ CTT CCT CCA TCT CAT AGC TGT C 3′ (SEQ ID NO: 2). In yet further embodiments, reverse primer that binds EGFR exon 8 is 5′ CTT CCT CCA TCT CAT AGC TGT C 3′. In yet further embodiments, the reverse primer that binds EGFR exon 8 has a sequence that is substantially identical to the sequence 5′ CTT CCT CCA TCT CAT AGC TGT C 3′.

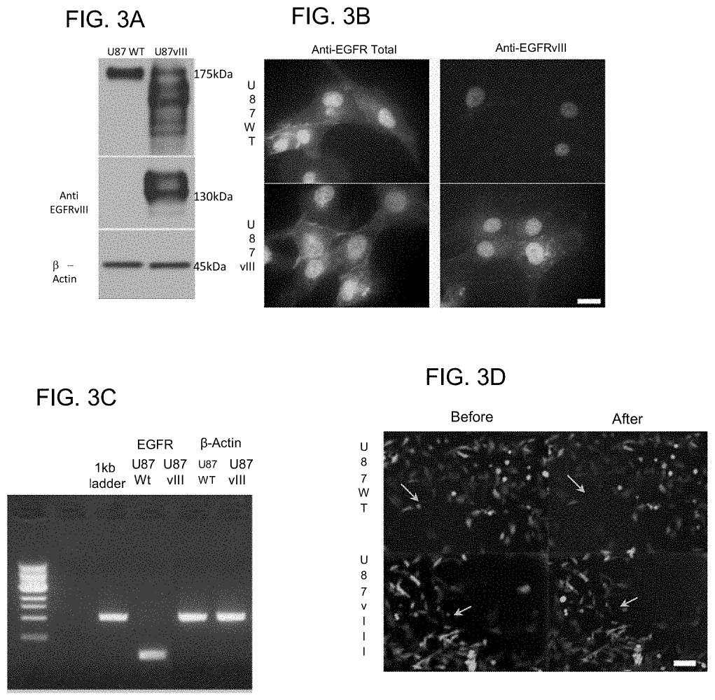

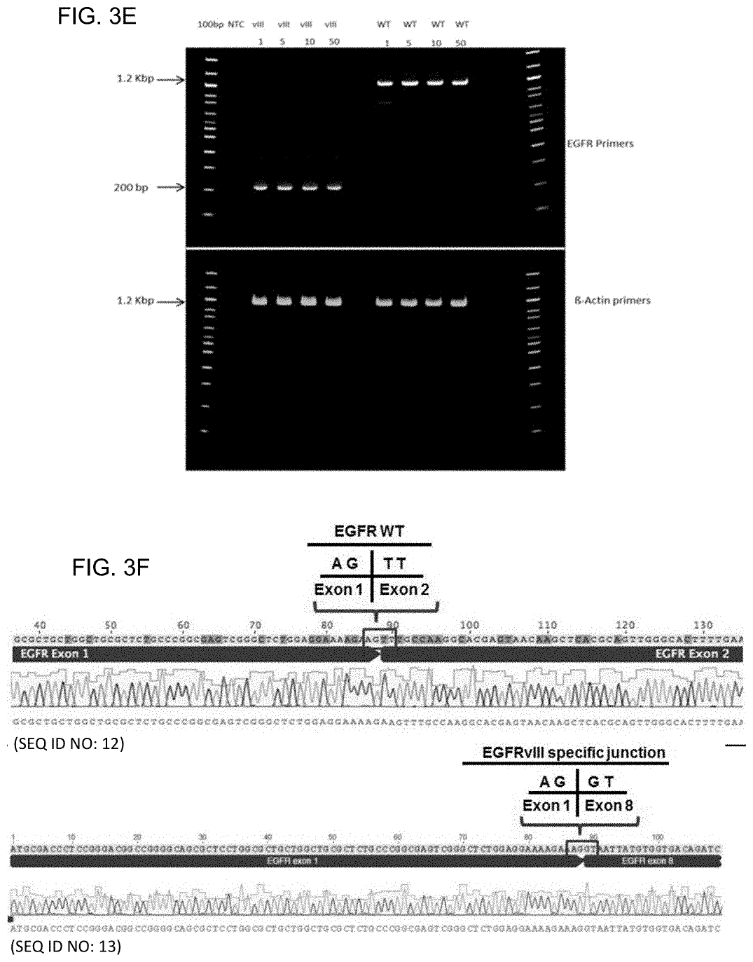

Provided herein are methods of detecting epidermal growth factor receptor - variant III (EGFRvIII) using a MGB probe that binds a junction formed by the joining of EGFR exons 1 and 8. This junction is depicted in FIG. 3F and results from an in-frame deletion of EGFR exons 2 through 7. In certain embodiments, the MGB probe includes an oligonucleotide having at least the sequence 5′ AAA GGT AAT TAT 3′ (SEQ ID NO: 3) that binds the junction of exons 1 and 8 present in EGFRvIII. In yet further embodiments, the MGB probe includes an oligonucleotide that is 5′ AAA GGT AAT TAT 3′. In certain embodiments, the primer and/or probes bind SEQ ID NO: 12, or a portion thereof.

In certain embodiments, EGFRvIII is detected with a limit of detection (LOD) of about 0.625×10⁻⁵ ng/ul. In certain embodiments, EGFRvIII is detected with a limit of quantification (LOQ) of about 0.003%.

In certain embodiments, EGFR variants R108K and/or A289V are detected with a limit of detection (LOD) of about 0.625×10⁻⁵ ng/ul. In certain embodiments, EGFR variants R108K and/or A289V are detected with a limit of quantification (LOQ) of about 0.003%. In certain embodiments, EGFR variants with R108K and/or A289V mutations are quantified at the level of one picogram in a background of six thousand picograms of EGFR wildtype. Provided herein are also methods of detecting or measuring levels of expression of wildtype EGFR in a biological sample using dPCR. The method includes contacting cDNA generated from the RNA present in a biological sample with a forward primer that binds EGFR exon 5, a reverse primer that binds EGFR exon 6, and a MGB probe having a oligonucleotide that binds the junction of EGFR exons 5 and 6. RNA present in a biological sample can be reversed transcribed to generate cDNA using routine methods and protocols known to one of skill in the art. Further, in certain embodiments, the cDNA generated from RNA present in the biological sample is amplified prior to performing dPCR. In certain embodiments, the MGB probe used for methods of detecting or measuring wildtype EGFR includes a fluorescent label (or dye), such as, for example, FAM, VIC, TET, and NED. In certain embodiments, the primer and/or probes bind SEQ ID NO: 13, or a portion thereof.

In certain embodiments, the forward primer that binds EGFR exon 5 includes the sequence 5′ AAG TGT GAT CCA AGC TGT CC 3′ (SEQ ID NO: 4). In yet further embodiments, the forward primer that binds EGFR exon 5 is 5′ AAG TGT GAT CCA AGC TGT CC 3′. In yet further embodiments, the forward primer that binds EGFR exon 1 has a sequence that is substantially identical to the sequence 5′ AAG TGT GAT CCA AGC TGT CC 3′.

In certain embodiments, the reverse primer that binds EGFR exon 6 includes the sequence 5′ TGC TGG GCA CAG ATG ATT T 3′ (SEQ ID NO: 5). In yet further embodiments, reverse primer that binds EGFR exon 6 is 5′ TGC TGG GCA CAG ATG ATT T 3′. In yet further embodiments, the reverse primer that binds EGFR exon 6 has a sequence that is substantially identical to the sequence 5′ TGC TGG GCA CAG ATG ATT T ′.

Provided herein are methods of detecting wildtype EGFR using an a MGB probe that binds a junction formed by the joining of EGFR exons 5 and 6. In certain embodiments, the MGB probe includes an oligonucleotide having at least the sequence 5′ AGG AGA ACT GCC 3′ (SEQ ID NO: 6) that binds the junction of exons 5 and 6 present in wildtype EGFR. In yet further embodiments, the MGB probe includes an oligonucleotide that is 5′ AGG AGA ACT GCC 3′.

The terms “nucleic acid” or “oligonucleotide,” as used herein, refer to at least two nucleotides covalently linked together. The depiction of a single strand also defines the sequence of the complementary strand. Thus, a nucleic acid also encompasses the complementary strand of a depicted single strand. Many variants of a nucleic acid can be used for the same purpose as a given nucleic acid. Thus, a nucleic acid also encompasses substantially identical nucleic acids and complements thereof. A single strand provides a probe that can hybridize to a target sequence under stringent hybridization conditions. Thus, a nucleic acid also encompasses a probe that hybridizes under stringent hybridization conditions.

Nucleic acids and oligonucleotides can be single stranded or double stranded, or can contain portions of both double stranded and single stranded sequences. The nucleic acid can be DNA, both genomic and cDNA, RNA, or a hybrid, where the nucleic acid can contain combinations of deoxyribo- and ribonucleotides, and combinations of bases including uracil, adenine, thymine, cytosine, guanine, inosine, xanthine hypoxanthine, isocytosine and isoguanine. Nucleic acids can be obtained by chemical synthesis methods or by recombinant methods. A particular nucleic acid sequence can encompass conservatively modified variants thereof (e.g., codon substitutions), alleles, orthologs, single nucleotide polymorphisms (SNPs), and complementary sequences as well as the sequence explicitly indicated.

Polymerase Chain Reaction (PCR) is an enzymatic reaction in which DNA fragments are synthesized and amplified from a substrate DNA in vitro. The reaction typically involves the use of two synthetic oligonucleotide primers, which are complementary to nucleotide sequences in the substrate DNA which are separated by a short distance of a few hundred to a few thousand base pairs, and the use of a thermostable DNA polymerase. The chain reaction consists of a series of 10 to 50 cycles. In each cycle, the substrate DNA is first denatured at high temperature. After cooling down, synthetic primers which are present in vast excess, hybridize to the substrate DNA to form double-stranded structures along complementary nucleotide sequences. The primer-substrate DNA complexes will then serve as initiation sites for a DNA synthesis reaction catalyzed by a DNA polymerase, resulting in the synthesis of a new DNA strand complementary to the substrate DNA strand. The synthesis process is repeated with each additional cycle, creating an amplified product of the substrate DNA.

The methods provided herein include performing digital PCR (dPCR). For purposes of this disclosure, dPCR includes any method that partitions an entire PCR reaction into smaller partitions, followed by submitting these individual partitions to enzymatic and cycling conditions well known in the art to perform PCR. Partitions may comprise oil droplet partitions, physical partitions on a chip, or a multi-well plate.

In some embodiments, digital PCR may be performed using the RainDrop platform (RainDance Technologies), QX-200 platform (BioRad), QuantStudio 3D (ThermoFisher) or the Biomark System (Fluidigm). Results are analyzed according to methods known in the art.

In certain embodiments, dPCR is performed using the QuantStudio 3D platform. For example, for the QuantStudio 3D platform, the reaction mixtures may include QuantStudio 3D digital PCR master mix V2 (Applied Biosystems), forward and reverse primers, and an MGB probe. For example, 900 nM of each forward and reverse primer for EGFR and 250 nM of the probe. In certain embodiments, loaded chips may be thermocycled as follows: 96° C. (e.g., for 10 minutes) followed by 45 cycles of 54° C. for 2 minutes and 98° C. for 30 seconds, and a final extension at 54° C. for 2 minutes, after which the reaction is held at 10° C.

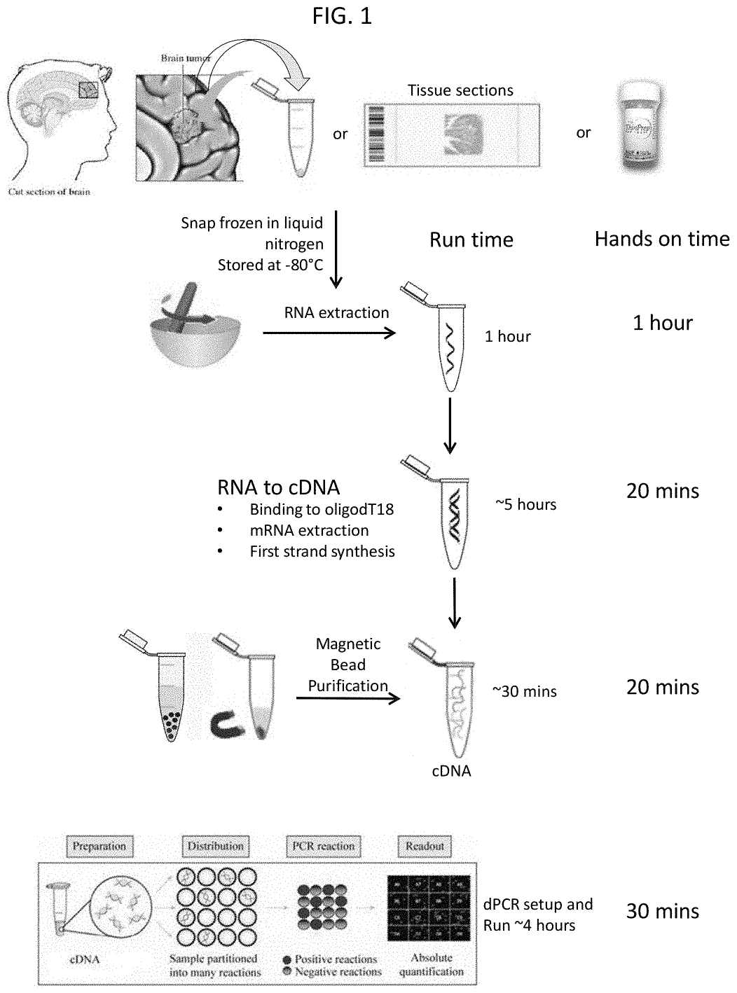

An exemplary workflow for detection of EGFR and/or EGFR variants is provided in FIG. 1. Accordingly, a biological sample is obtained from a subject. The sample may be a surgically isolated tissue, a tissue section (e.g., FFPE slides), or a preserved tissue sample (e.g., a ThinPrep® sample or sample persevered in PreservCyt®). In certain embodiments, the isolated sample is frozen, for example, by snap freezing in liquid nitrogen or storing at −20° C. or -80° C. RNA is extracted from the biological sample using methods or kits available to one of skill in the art. The step of isolating RNA from a biological sample can take about 1 hour or less. Subsequently, RNA is reverse transcribed to generate cDNA, for example, using oligo(dT)18 primers. The process of generating cDNA sample from RNA can take about 5 hours, but may be accomplished in less time, for example, as little as 1 hour or 30 minutes. cDNA obtained from a sample can be further isolated or purified using various approaches (e.g., using magnetic-bead based purification). Isolation and purification of cDNA can be performed in about 30 minutes. The cDNA obtained from a biological sample is combined with reagents to perform dPCR. The process of setting up dPCR (including, e.g., mixing reagents, loading chips, and/or distributing samples) can be accomplished in about 30 minutes, while dPCR amplification and detection takes about 4 hours, but can be accomplished in less time depending on, for example, the device, programmed times, and conditions. Total assay time is less than twenty-four hours.

“Primer” as used herein, refers to an oligonucleotide capable of acting as a point of initiation for DNA synthesis under suitable conditions. Suitable conditions include those in which hybridization of the oligonucleotide to a template nucleic acid occurs, and synthesis or amplification of the target sequence occurs, in the presence of four different nucleoside triphosphates and an agent for extension (e.g., a DNA polymerase) in an appropriate buffer and at a suitable temperature.

“Probe” or “MGB probe” as used herein refers to a sequence-specific oligonucleotide having an attached fluorescent label, quencher, and minor groove binder.

“Quencher” refers to a molecule or part of a compound, which is capable of reducing the emission from a fluorescent donor when attached to or in proximity to the donor. Quenching may occur by any of several mechanisms including fluorescence resonance energy transfer, photo-induced electron transfer, paramagnetic enhancement of intersystem crossing, Dexter exchange coupling, and exciton coupling such as the formation of dark complexes.

As used herein, the term “attached”, particularly with respect to a probe, can refer to an indirect or direct linkage of elements. For example, two elements of a probe, such as an oligonucleotide and a fluorescent label may be attached by direct conjugation or may be attached, or linked, indirectly by one or more intervening elements of the probe (e.g., another fluorescent label, MGB, quencher, oligonucleotide, or spacer sequence). In certain embodiments, elements of a probe are each covalently joined to a spacer sequence, and as a result are attached to one another.

The methods described herein include MGB probes that are labeled for use or detection in a dPCR assay. In certain embodiments, a probe is labeled with a dye selected from FAM, VIC, TET, and NED.

In addition to an oligonucleotide, minor groove binder, a fluorescent label (dye) and quencher, the probes described herein may include spacer sequences that separate one or more of these elements. The spacer sequences can be of varying lengths and will in some embodiments be specific to a particular target sequence to which other domains of the ligation probe (e.g., the oligonucleotide). In certain embodiments, the probe includes one or more spacer sequences that are each 1, 2, 3, 4, 5, 6, 7, 8, 9, or 10 nucleotides in length.

“Complement” or “complementary” is used herein to refer to a nucleic acid may mean Watson-Crick (e.g., A-T/U and C-G) or Hoogsteen base pairing between nucleotides or nucleotide analogs of nucleic acid molecules. A full complement or fully complementary means 100% complementary base pairing between nucleotides or nucleotide analogs of nucleic acid molecules. In some embodiments, the complementary sequence has a reverse orientation (5′-3′).

“Substantially identical”, as used herein, means that a first and a second sequence are at least 60%, 65%, 70%, 75%, 80%, 85%, 90%, 95%, 97%, 98% or 99% identical over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides or amino acids, or with respect to nucleic acids, if the first sequence is substantially complementary to the complement of the second sequence.

“Detection,” “detect”, “detecting” and grammatical variations thereof refer to identifying the presence of a gene, such as EGFR or an EGFR variant (e.g. EGFRvIII), or the expression levels thereof, in a sample. Detection also means detecting the absence of a gene or variant. Thus, detection means determining the level of a gene or variant, either quantitatively or qualitatively.

As used herein, “minor groove binder” refers to small molecules that fit into the minor groove of double-stranded DNA, typically in a sequence-specific manner. Minor groove binders may be long, flat molecules that can adopt a crescent-like shape and thus fit snugly into the minor groove of a double helix, often displacing water. Minor groove binding molecules may typically comprise several aromatic rings connected by bonds with torsional freedom such as furan, benzene, or pyrrole rings. Minor groove binders may be antibiotics such as netropsin, distamycin, berenil, pentamidine and other aromatic diamidines, Hoechst 33258, SN 6999, aureolic anti-tumor drugs such as chromomycin and mithramycin, CC-1065, dihydrocyclopyrroloindole tripeptide (DPI₃), 1,2-dihydro-(3H)-pyrrolo [3,2-e]indole-7-carboxylate (CDPI3), and pyrrole-imidazole polyamides (Gottesfeld, J. M., et al., J. Mol. Biol. (2001) 309:615-629), and related compounds and analogues, including those described in Nucleic Acids in Chemistry and Biology, 2nd ed., Blackburn and Gait, eds., Oxford University Press, 1996, and PCT Published Application No. WO 03/078450, the contents of which are incorporated herein by reference. Minor groove binders may increase the T. of the primer or a probe to which they are attached, allowing such primers or probes to effectively hybridize at higher temperatures.

“Gene”, as used herein, may be a natural (e.g., genomic) or synthetic gene comprising transcriptional and/or translational regulatory sequences and/or a coding region and/or non-translated sequences (e.g., introns, 5′- and 3′-untranslated sequences). The coding region of a gene may be a nucleotide sequence coding for an amino acid sequence or a functional RNA, such as tRNA, rRNA, catalytic RNA, siRNA, miRNA or antisense RNA. A gene may also be an mRNA or cDNA corresponding to the coding regions (e.g., exons and miRNA) optionally comprising 5′- or 3′-untranslated sequences linked thereto. A gene may also be an amplified nucleic acid molecule produced in vitro, comprising all or a part of the coding region and/or 5′- or 3′-untranslated sequences linked thereto. “Sensitivity” as used herein, refers to a measure of how well the method or assay (including, e.g., the particular probes, primers, and or/or PCR conditions) result in the correct identification or detection of a gene target.

As used herein, the term “wildtype” sequence refers to a coding, a non-coding or an interface sequence which is an allelic form of sequence that performs the natural or normal function for that sequence. Wildtype sequences include multiple allelic forms of a cognate sequence, for example, multiple alleles of a wild type sequence may encode silent or conservative changes to the protein sequence that a coding sequence encodes. A wildtype EGFR sequence is identified, for example, in NCBI's RefSeq database accession NM_005228 (EGFR transcript variant 1 sequence).

Provided herein are methods for treating a subject with a cancer that include detection of EGFR and/or EGFRvIII in a biological sample from the subject. Accordingly, in certain embodiments, cDNA is generated from RNA in a biological sample obtained from a subject and dPCR is performed to detected EGFR and/or EGFRvIII. In certain embodiments, the methods of treatment included detection of EGFRvIII using a forward primer that binds EGFR exon 1, a reverse primer that binds EGFR exon 8, and a minor groove binder (MGB) probe having an oligonucleotide comprising at least the sequence of: 5′ AAA GGT AAT TAT 3′ (SEQ ID NO: 3) that binds the junction of EGFR exon 1 and 8. In certain embodiments, the presence of amplified sequences indicates a cancer associated with EGFRvIII and the subject is subsequently administered a treatment to reduce, inhibit, and/or delay the progression of a cancer or a tumor in the subject. In certain embodiments, the treatment includes a therapeutic agent that targets EGFRvIII. In certain embodiments, the treatment includes administering an immunotherapy, such as chimeric antigen receptor (CAR) T cells that are specific for EGFRvIII. In certain embodiments, the subject is identified as having EGFRvIII-positive GBM based on detection of EGFRvIII in a biological sample obtained from the subject. In yet a further embodiment, the patient diagnosed with cancer is stratified based on the detection of EGFRvIII. In certain embodiments, the subject is diagnosed with an EGFRvIII-positive GBM and administered a treatment for GBM or an EGFRvIII-positive cancer.

In certain embodiments, the presence of amplified sequences indicates a cancer associated with EGFR variants R108K and/or A289V and the subject is subsequently administered a treatment to reduce, inhibit, and/or delay the progression of a cancer or a tumor in the subject. In certain embodiments, the treatment includes a therapeutic agent that targets the EGFR variant(s). In certain embodiments, the treatment includes administering an immunotherapy, such as chimeric antigen receptor (CAR) T cells that are specific for the EGFR variants R108K and/or A289V. In certain embodiments, the subject is identified as having EGFR variant R108K and/or A289V -positive GBM based on detection of one or both of these EGFR variants in a biological sample obtained from the subject. In yet a further embodiment, the patient diagnosed with cancer is stratified based on the detection of one or both of these EGFR variants. In certain embodiments, the subject is diagnosed with an EGFR variant R108K and/or A289V - positive GBM and administered a treatment for GBM or an EGFR variant R108K and/or A289V-positive cancer.

In certain embodiments, methods of treatment are provided that include detection of wildtype EGFR in a subject that has been diagnosed with or is suspected of having cancer. Accordingly, a subject may be identified as having cancer in which the EGFR gene is amplified or overexpressed. The method of treatment includes contacting cDNA generated from the RNA present in a biological sample with a forward primer that binds EGFR exon 5, a reverse primer that binds EGFR exon 6, and a MGB probe having an oligonucleotide comprising at least the sequence of: 5′ AGG AGA ACT GCC 3′ (SEQ ID NO: 6) that binds the junction of EGFR exons 5 and 6. In certain embodiments, the detection of increased levels of EGFR expression relative to, for example, a healthy control, identifies a cancer or tumor that has amplification or overexpression of EGFR. In certain embodiments, the subject identified as having a cancer or tumor with amplification or overexpression of EGFR is treated for the cancer.

In certain embodiments, provided herein the cancer is glioblastoma and the method comprises treatment of a subject identified as having amplification or overexpression of EGFR and/or EGFRvIII-positive glioblastoma. In certain embodiments, the treatment for glioblastoma includes administering temozolomide.

As used herein, “treating” or “treat” describes the management and care of a patient for the purpose of combating a disease, condition, or disorder and includes, but is not limited to, the administration of chemotherapy, immunotherapy, radiotherapy, or a combination thereof, to alleviate the symptoms or complications of a disease, condition or disorder, or to eliminate the disease, condition or disorder. Treatments for cancer include, but are not limited to, the administration of chemotherapeutic agents, the administration of anti-cancer agents, radiation treatment, immunotherapy, surgery, radiation therapy, targeted therapy, hormone therapy and stem cell transplant. In certain embodiments, treatments is combined with surgery, for example surgery followed by radiation and/or chemotherapy. Cancer treatment can include administering to the subject a therapeutically effective dose of at least one class of drugs. The terms “effective amount” and “therapeutically effective amount” of a drug, agent or compound of the invention is meant a nontoxic but sufficient amount of the drug, agent or compound to provide the desired effect, for example, a response or benefit in the subject.

Chemotherapeutic agents can include cyclophosphamide, fluorouracil (or 5-fluorouracil or 5-FU), methotrexate, thiotepa, carboplatin, cisplatin, taxanes, paclitaxel, protein-bound paclitaxel, docetaxel, vinorelbine, tamoxifen, raloxifene, toremifene, fulvestrant, gemcitabine, irinotecan, ixabepilone, temozolmide, topotecan, vincristine, vinblastine, eribulin, mutamycin, capecitabine, capecitabine, anastrozole, exemestane, letrozole, leuprolide, abarelix, buserlin, goserelin, megestrol acetate, risedronate, pamidronate, ibandronate, alendronate, denosumab, zoledronate, trastuzumab, tykerb or bevacizumab, or combinations thereof.

In certain embodiments, the method includes administering a therapeutically effective amount of a cancer immunotherapy. Non-limiting examples of cancer immunotherapies include vaccines (e.g., peptide vaccines), antibodies, chimeric antigen receptor T cells (CAR-T cells), and combinations thereof. In particular embodiments, the antigens presented by infected or cancerous cells are highly specific to each disease or condition, and the vaccines, antibodies, and/or CAR-T cells used is dependent on the disease or condition being treated. In certain embodiments, the methods include administering an antibody molecule that specifically binds PD-1, PD-L1, CTLA-4, CD19, CD20, BAFFR, TACI, EGFR or a variant, APRILR, or BCMA.

In certain embodiments, the treatments includes administering a tyrosine kinase inhibitor to a subject. Exemplary tyrosine kinase inhibitors include, but are not limited to, erlotinib (Tarceva); gefitinib (Iressa); imatinib (Gleevec); sorafenib (Nexavar); sunitinib (Sutent); trastuzumab (Herceptin); bevacizumab (Avastin); rituximab (Rituxan); lapatinib (Tykerb); cetuximab (Erbitux); panitumumab (Vectibix); everolimus (Afinitor); alemtuzumab (Campath); gemtuzumab (Mylotarg); temsirolimus (Torisel); pazopanib (Votrient); dasatinib (Sprycel); nilotinib (Tasigna); vatalanib (Ptk787; ZK222584); CEP-701; SU5614; MLN518; XL999; VX-322; Azd0530; BMS-354825; SKI-606 CP-690; AG-490; WHI-P154; WHI-P131; AC-220; or AMG888. Tyrosine kinase inhibitors can include, but are not limited to, epidermal growth factor receptor (EGFR) inhibitors. Exemplary EGFR inhibitors include, but are not limited to, gefitinib (Iressa); lapatinib (Tykerb); cetuximab (Erbitux); erlotinib (Tarceva); panitumumab (Vectibix); PKI-166; canertinib (CI-1033); matuzumab (Emd7200) or EKB-569. EGFR inhibitors can include, but are not limited to, pan-human epidermal growth factor receptor (pan-HER) inhibitors. Pan-HER inhibitors can include, but are not limited to Dacomitinib, afatinib, neratinib.

Provided herein are methods for detection of EGFR missense mutations, including R108K and A289V. The methods include detection of EGFR variants in a biological sample using dPCR conditions provided herein. In certain embodiments, the conditions include an initial stage of 45° C. for 1 min and 98° C. for 2 minutes, 30 seconds. In certain embodiments, the initial stage is followed by 40 or about 40 cycles of 95° C. for 2 minutes, 49.5° C. for 10 seconds, and 53.5° C. for 30 seconds. Methods of detection of EGFR point mutations, including suitable dPCR probes and primers are known in the art and are described, for example, by Illei et al. Clinical mutational profiling of 1006 lung cancers by next generation sequencing, Oncotarget. 2017 Nov. 14; 8(57): 96684-96696, which is incorporated by reference herein.

In certain embodiments, provided herein are kits for performing detection of EGFR and EGFR variants. In certain embodiments, the kit comprises a forward primer that binds EGFR exon 1, a reverse primer that binds EGFR exon 8, and a MGB probe comprising an oligonucleotide having at least the sequence of: 5′ AAA GGT AAT TAT 3′ (SEQ ID NO: 3) that binds the junction of EGFR exons 1 and 8. In certain embodiments, the kit comprises a forward primer that binds EGFR exon 5, a reverse primer that binds EGFR exon 6, and a MGB probe comprising an oligonucleotide having at least the sequence of: 5′ AGG AGA ACT GCC 3′ (SEQ ID NO: 6) that binds the junction of EGFR exons 5 and 6. In certain embodiments, the forward primer that binds EGFR exon 1 comprises 5′ TCG GGC TCT GGA GGA AA 3′ (SEQ ID NO: 1) and/or the reverse primer that binds EGFR exon 8 comprises 5′ CTT CCT CCA TCT CAT AGC TGT C 3′ (SEQ ID NO: 2). In certain embodiments, the forward primer that binds EGFR exon 5 comprises 5′ AAG TGT GAT CCA AGC TGT CC 3′ (SEQ ID NO: 4) and/or the reverse primer that binds EGFR exon 6 comprises 5′ TGC TGG GCA CAG ATG ATT T 3′(SEQ ID NO: 5). In certain embodiments, the kit includes additional reagents, including those suitable for processing a tissue sample, nucleic acid extraction from a biological sample, performing a reverse transcription reaction, and/or performing dPCR.

With regard to the description of these inventions, it is intended that each of the compositions herein described, is useful, in another embodiment, in the methods of the invention. In addition, it is also intended that each of the compositions herein described as useful in the methods, is, in another embodiment, itself an embodiment of the invention.

Unless defined otherwise in this specification, technical and scientific terms used herein have the same meaning as commonly understood by one of ordinary skill in the art to which this invention belongs and by reference to published texts, which provide one skilled in the art with a general guide to many of the terms used in the present application.

“Comprising” is a term meaning inclusive of other components or method steps. When “comprising” is used, it is to be understood that related embodiments include descriptions using the “consisting of” terminology, which excludes other components or method steps, and “consisting essentially of” terminology, which excludes any components or method steps that substantially change the nature of the embodiment or invention. It should be understood that while various embodiments in the specification are presented using “comprising” language, under various circumstances, a related embodiment is also described using “consisting of” or “consisting essentially of” language.

It is to be noted that the term “a” or “an”, refers to one or more, for example, “a probe”, is understood to represent one or more probes. As such, the terms “a” (or “an”), “one or more,” and “at least one” is used interchangeably herein.

As used herein, the term “about” means a variability of plus or minus 10% from the reference given, unless otherwise specified.

### EXAMPLES

The invention is now described with reference to the following examples. These examples are provided for the purpose of illustration only and the invention should in no way be construed as being limited to these examples but rather should be construed to encompass any and all variations that become evident as a result of the teaching provided herein.

### Example 1

### Materials and Methods

### Cell Culture

U87 and U87 EGFRvIII were provided by Dr. Laura Johnson (University of Pennsylvania, Philadelphia, Pa.), and maintained in Improved MEM supplemented with 10% fetal bovine serum (FBS, Invitrogen, Carlsbad, Calif.), 1× HEPES, and 1.0% Penicillin-Streptomycin. Patient-derived glioma stem cells lines NS039, HK296, HK248, and HK301 were obtained from Dr. Harley Kornblum (UCLA, Los Angeles, Calif.), and maintained in DMEM/F12 1:1 containing 1.0% Penicillin-Streptomycin, 1× B-27 with vitamin A, 1 mM pyruvate, 50 ng/ml Epidermal Growth Factor (EGF, Peprotech, Rocky Hill, N.J.), 20 ng/ml Fibroblast Growth Factor (FGF, Peprotech, Rocky Hill, N.J.), 5 μg/ml Heparin Sulfate (Sigma Aldrich). The patient derived glioma stem cell line T4213 was obtained from Dr. Yi Fan (UPenn) and maintained in Neurobasal A media supplemented with 1.0% Penicillin-Streptomycin, 0.5× B-27 without vitamin A (Invitrogen), 1 mM sodium pyruvate, 1× Glutamate, 20 ng/ml EGF (Peprotech, Rocky Hill N.J.) and 20 ng/ml FGF (Peprotech). All cells were cultured in a humidified incubator maintained at 37° C. and 5% CO₂. Cell lines were certified mycoplasma free by the MycoAlert® Assay (Cambrex) on a regular basis.

### Isolation of Cells by Microcapillary Aspiration

The isolation of selected cells was performed using a capillary-based vacuum-assisted cell acquisition system (Kuigpick™, NeurolnDx, Signal Hill, Calif.).¹⁷ Calibration was initially performed under bright field conditions, followed by the collection of individual fluorescent cells via micro-capillary aspiration under fluorescence microscopy. Single cells (U87 glioma cells or patient derived GSCs) were collected in cell lysis buffer (0.2% Triton X-100 and 2 units of RNAse OUT).

### Control Blood Spike Experiment

All animal experiments were approved by the Institute for Animal Care and Use Committee at the University of Pennsylvania. Mouse blood was collected via terminal cardiac puncture into a three milliliter syringe containing 250 μL of acid citrate dextrose. One thousand cells of patient-derived GSCs were then spiked into the collected mouse blood. Spiked cells were processed through a CelSee microfluidic chip (CelSee, Plymouth Mich., USA) following the manufacturer's instructions, and then plated into single wells of an eight-well chamber slide using DMEM media. Cells were incubated with an adenovirus probe (Oncolys BioPharma, Fort Lee, N.J.) for 24 h at 37° C. This adenovirus probe utilizes telomerase expression to drive replication of an adenovirus that contains a GFP reporter. The chamber wells were then imaged via a computer-driven semi-automated fluorescence microscope, with subsequent analysis performed via Image Pro Plus (Media Cybernetics, Rockville, Md.). Analysis included sorting and filtering based on reproducible parameters such as fluorescent intensity (two and half standard deviations above background mean), cell diameter (between 7 and 70 μm), and absence of clumping or debris.¹⁸

### Flank Tumor Implantation

NS039 cells were resuspended in PBS, and then 2.0×10⁶ cells were injected into the right flank of six week old female athymic nude mice (Charles River Labs). Mice were humanely euthanized, and tumor tissue was collected following seven weeks of growth.

### Intracranial Tumor Implantation

U87 overexpressing EGFRvIII were modified to stably express mCherry and firefly luciferase (Genecopoeia, Rockville Md.). Patient derived T4213 GSCs were modified to stably express green fluorescent protein and firefly luciferase (Genecopoeia, Rockville Md.). Cells were dissociated into single cell suspensions using TrypLE Express (Life Technologies), and 5×10⁵ cells were resuspended in PBS and stereotactically implanted into the right striatum of the brains of six- to eight-week-old athymic nude mice (Charles River Laboratories). Tumor growth was monitored weekly by bio-luminescence (BLI), and mice were humanely sacrificed after four to six weeks post implantation or when neurological defects became significant. The brain was removed and tumor tissue was dissected for histologic or molecular analysis.

### Patient Samples

Minute portions of human patient glioblastoma were obtained fresh immediately following intra-operative diagnosis of high-grade glioma. Diagnosis of glioblastoma was confirmed by a board-certified neuropathologist (MPN). Use of human tumor tissue was approved by an independent institutional review board at the Hospital of the University of Pennsylvania (HUP IRB protocol 827290).

Patients samples were also obtained from the Penn Neurosurgery Tumor Tissue Bank under a University of Pennsylvania Institutional Review Board approved protocol.

Samples were de-identified and associated with sequencing results obtained from the University of Pennsylvania's Center for Personalized Diagnostics.

### Sample Preparation and Workflow

Entire workflow is illustrated in FIG. 1, with each step described further below.

Total RNA Extraction from Tumor Materials

Mouse and patient derived tumor tissue were snap frozen in liquid nitrogen and stored at −80° C. Twenty-one patient tumor samples were also collected in PreservCyt® to compare the stability of tissue for RNA extraction in PreservCyt media. Tumor tissue was ground with a chilled mortar and pestle to a fine powder and transferred to a collection tube. Total RNA was collected using the Nucleospin RNA isolation kit (Macherey-Nagel Germany) according to the manufacturer's instruction. RNA was extracted from Fresh Frozen Paraffin Embedded (FFPE) patient samples as well as from frozen sections using RecoverALL™ Total Nucleic Acid Isolation kit (Invitrogen, Waltham Mass., USA) following the manufacturer's protocol.

cDNA Extraction from Tumor Materials

Total RNA from tumor material was extracted and reversed transcribed to cDNA, which was subsequently amplified by PCR as described by Picelli et al 2014.¹⁹ The Poly(A) tail was hybridized with OligodT primer (5′-AAGCAGTGGTATCAACGCAGAGTACT₃₀VN-3′) (SEQ ID NO: 7) by incubating 2 μL of total RNA with 1 μL of dNTP mix and 1 μL of oligodt30VN (100 μM) at 72° C. for three minutes. Reverse Transcriptase mix was prepared with 5M Betaine and Locked nucleic acid modified TSO (5′-AAGCAGTGGTATCAACGCAGAGTACATrGrG+G-3′) (SEQ ID NO: 8) and the reaction was carried out in a thermal cycler with a heated lid for 90 mins at 42° C. followed by 10 cycles at 50° C. for 2 mins and 42° C. for 2 mins. cDNA was further pre-amplified using 10 μM of random IS PCR primer (5′-AAGCAGTGGTATCAACGCAGAGT-3′) (SEQ ID NO: 9) with KAPA HiFi Hotstart Ready Mix (2×) (Kapa Biosystems, Wilmington Mass. USA) and thermocycled at following conditions: denaturation at 98° C. for 3 minutes, followed by 18 cycles at 98° C. for 20 secs, 67° C. for 15 secs, extension at 72° C. for 6 minutes, final extension at 72° C. for 5 minutes. PCR products were purified using Agencourt® AMPure®XP kit (Beckman Coulter, Brea, Calif.). cDNA concentration was determined using Qubit 3 fluorimeter utilizing Qubit dsDNA High Sensitivity Assay kit (Life Technologies Corporation, Eugene, Oreg.). cDNA was diluted further to a concentration of 10 ng/μL before proceeding for dPCR assay.

### Digital PCR Probe for EGFRvIII and EGFR WT Detection

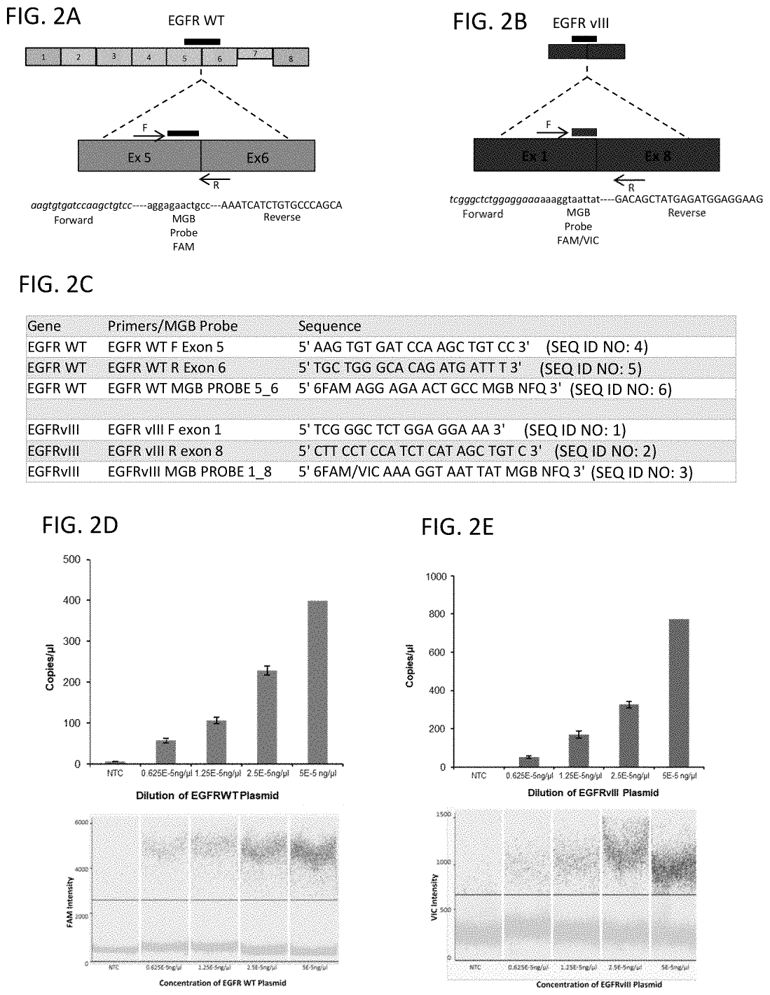

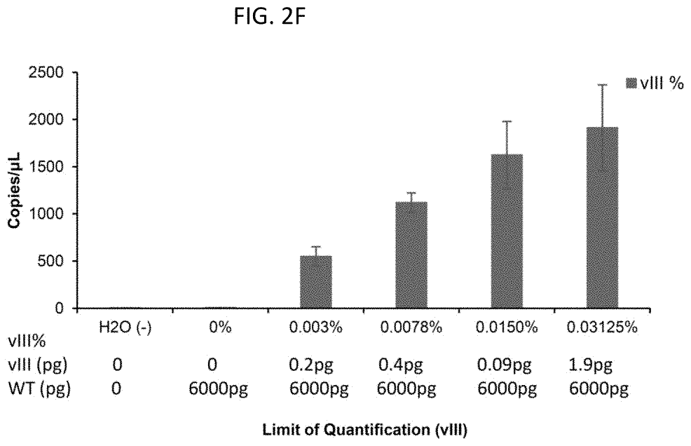

Primers and Minor Groove Binder (MGB) probe for EGFRvIII were designed such that the MGB probe recognizes the junction of exon 1 and exon 8. Forward and Reverse primers are located on exon 1 and exon 8 respectively (FIG. 2B). Forward and reverse primers and MGB probe for EGFR WT are located on exon 5, exon 6, and at the junction of exon 5 and 6 respectively (FIG. 2A). The EGFR WT MGB probe was fluorescently labeled with fluorescein amidite (FAM), and the EGFRvIII MGB probe was fluorescently labeled with either FAM or VIC. Gene copies per microliter were normalized by running RNaseP (Thermofisher, Foster City Calif.) on the same chip with either the EGFRWT or EGFRvIII reaction mix.

The digital PCR reaction were carried out using the Quantstudio 3D digital PCR master mix V2 (Applied Biosystems, Foster City, Calif.) following the manufacturer's instructions using 900 nM of each primer pair, and 250 nM of the MGB probe. The Quantstudio 3D digital PCR chip was loaded and sealed thus avoiding cross contamination between reaction wells. The chips were thermo-cycled using ProFlex PCR system (Applied Biosystem, Life Technologies). Cycling conditions were as follows: one cycle of 96° C. for 10 minutes, followed by 45 cycles of 54° C. for 2 minutes, 98° C. for 30 seconds, final extension at 54° C. for 2 minutes, followed by an infinite hold at 10° C. Individual chips were read on a Quantstudio 3D Digital PCR chip reader. For each chip the instrument generates a single .eds file that contains the processed image data, and results from a preliminary analysis. The data is then transferred onto ThermoFisher cloud for further analysis by proprietary software.

### Calculation of EGFRvIII in Tumor Samples

Tumor samples collected from GBM patients were analyzed for EGFR WT and EGFRvIII. For every tumor sample, two chips were run separately with the same concentration of cDNA containing EGFRvIII primers and probe and EGFRWT (FAM labeled) with RNaseP (VIC labeled) respectively.²⁰ EGFRWT and EGFRvIII copies/μL were normalized to the RNAseP internal control. Amplified EGFR was considered positive if the ratio of EGFR WT to RnaseP is ≥1.5.²⁰ The percentage of EGFRvIII was calculated by

$$\frac{vIII copies \text{/} µL}{vIII copies \text{/} µL + WTcopies \text{/} µL}$$

and multiplied by one hundred.

### Western Blot

Cells were lysed in the presence of RIPA buffer (Thermo Scientific) supplemented with 1 mM PMSF, 1 mM Sodium orthovanadate, 1× complete MINI (Roche). Protein concentration was determined using BSA standard curve and Pierce ® 660 nM reagent. Denatured protein was then loaded on 4-12% Bis-Tris gel (Invitrogen), and subsequently transferred onto PVDF membrane. Membranes were blocked in non-fat milk followed by incubation in primary antibody overnight at 4° C. Secondary antibody incubation was for one hour at room temperature. All antibodies were from Cell Signaling (Danvers, Mass.). Immunoreactive bands were visualized via enhanced chemiluminescence by incubating membranes with the ECLTM Western Blotting Detection Reagent in accordance with the manufacturer's recommendations (GE Healthcare Bio-Sciences, Pittsburgh, Pa.). Immunofluorescence

Cells (1.5×10³) were plated onto a chamber slide and allowed to grow overnight. Cells were then fixed using one percent formalin in PBS for 10 minutes, followed by permeabilization using 0.25% Triton X-100 for 10 minutes at room temperature. After blocking with 10% serum for one hour, slides were incubated with primary antibodies overnight at 4° C., followed by an incubation with Alexa Fluor® 495 conjugated goat anti-rabbit IgG (H+L) (Invitrogen), for one hour at room temperature.

### Semi-Quantitative PCR to Confirm Presence of WI Mutation

cDNA amplified from glioma cells (both U87 and GSC) was reverse transcribed using the following primers: 5′-ATGCGACCCTCCGGGACG (forward, exon 1) (SEQ ID NO: 10) and 5′-ATTCCGTTACACACTTTGCGGC (reverse, exon 9) (SEQ ID NO: 11). Expected amplicon sizes with these primers were ˜1000 bp for EGFR WT cDNA and ˜200 bp for EGFRvIII cDNA. cDNA was then resolved on a TBE gels and stained with SYBR safe DNA stain (Invitrogen, Carlsbad, Calif.). Bands were visualized using the ChemiDoc imaging system (Bio-Rad, Hercules, Calif.).

### Statistical Analysis

Statistical significance was performed in GraphPad Prism 7 using a one-way ANOVA followed by post-hoc Tukey's test. P-values less than 0.05 were considered significant.

### Example 2

### EGFRvIII Detection Assay

We developed a sensitive dPCR assay that is capable of detecting the variant III of EGFR in biological samples, including patient derived glioma neurosphere cell lines, orthotopically implanted tumor cells, and from patient-derived tumor specimens (fresh, frozen, and paraffin embedded). Our assay utilizes an EGFRvIII MGB probe and primers that recognize the unique sequence generated by the fusion of exon 1 and exon 8 (FIG. 2B). The assay leverages around approximately twenty thousand individualized reactions of dPCR to identify rare mutations from background DNA. Furthermore, the assay is able to detect EGFRvIII in a wide variety of specimens in less than twenty-four hours. The assay duplicates the findings of NGS and IHC in patient derived tumor specimens, and in certain cases detects EGFRvIII when NGS and IHC cannot.

The newly developed probes and primers were optimized and validated using either plasmid DNA containing the wildtype EGFR sequence or EGFRvIII sequence (FIG. 2D and FIG. 2E). Next, we determined the Limit of Detection (LOD) and Limit of Quantification (LOQ) of our assay. The LOD was defined as the lowest detectable concentration of EGFRvIII. The LOQ referred to the lowest detectable ratio of EGFRvIII to EGFR wildtype. Utilizing EGFRvIII plasmid DNA, we were able to determine that the LOD of this assay was 0.625×10⁻⁵ ng/μL. In a mixture of EGFRvIII and wildtype plasmid DNA, the LOQ for our assay was 0.003% (FIG. 2F). Copies per microliter of EGFRvIII detected in samples containing 0.003% vIII was found to be statistical significant (p<0.0001) when compared to water only and 0% vIII samples.

We utilized U87 cells over-expressing the EGFRvIII (U87 vIII) to demonstrate the ability of our assay to detect the EGFRvIII mutation from cell lines. Immunoblot and immunofluorescence analysis qualitatively confirmed the presence of the mutated EGFRvIII receptor in U87vIII when compared to wildtype control cells (FIG. 3A and FIG. 3B). This confirmatory step allowed us to move forward with PCR analysis of these cell lines. We used conventional PCR to detect the vIII sequence in DNA extracted from U87vIII cell lines using gene specific primers (FIG. 3C). Direct Sanger sequencing was used to detect the EGFRvIII specific junction between exon 1 and exon 8 from isolated U87vIII cells. In order to perform Sanger sequencing, pure populations of U87 vIII cells were isolated from a mixed population of U87 WT and U87 vIII using the Kuiqpick system (FIG. 3D). Kuiqpick isolation allowed us to work with smaller numbers of clonally distinct cells, and extract cDNA at higher concentrations. Extracted cDNA was amplified using gene specific primers for EGFR and then sequenced (FIG. 3E). The EGFR transcript variant 1 (accession NM_005228) sequence was acquired from NCBI's RefSeq database. Sequence readings were trimmed and aligned to the reference using Geneious version 7.1.8. Representative sequencing experiments for the single EGFRvIII clones are shown in FIG. 3F.

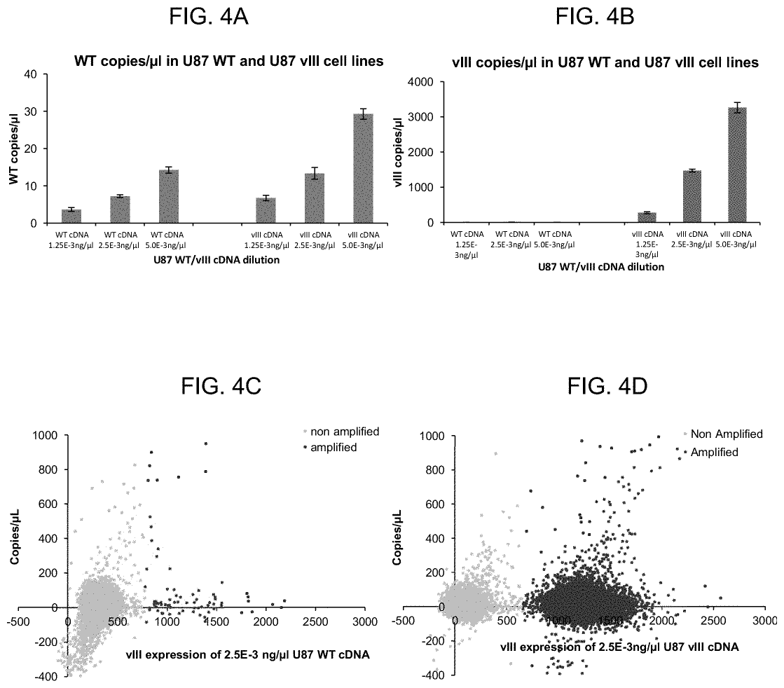

Following these confirmatory steps, our dPCR assay was run on cDNA extracted from both U87 WT and U87 vIII. The concentrations of cDNA tested ranged from 1.25×10⁻³ ng/μL to 5×10⁻³ ng/μL. The EGFR WT probe and primers were able to detect expression of the wildtype isoform of EGFR in both U87 WT and U87vIII at all concentrations (FIG. 4A). Similarly, the EGFRvIII probe and primer mixture was able to detect vIII in U87vIII cDNA, but not in U87WT cDNA, confirming the specificity of our probe and primer mixture in cDNA extracted from cells (FIG. 4B). FIG. 4C and FIG. 4D represent the scatter plots of vIII expression on U87 WT cDNA (FIG. 4C) and U87 vIII cDNA (FIG. 4D) at a concentration of 2.5×10⁻³ ng/μL.

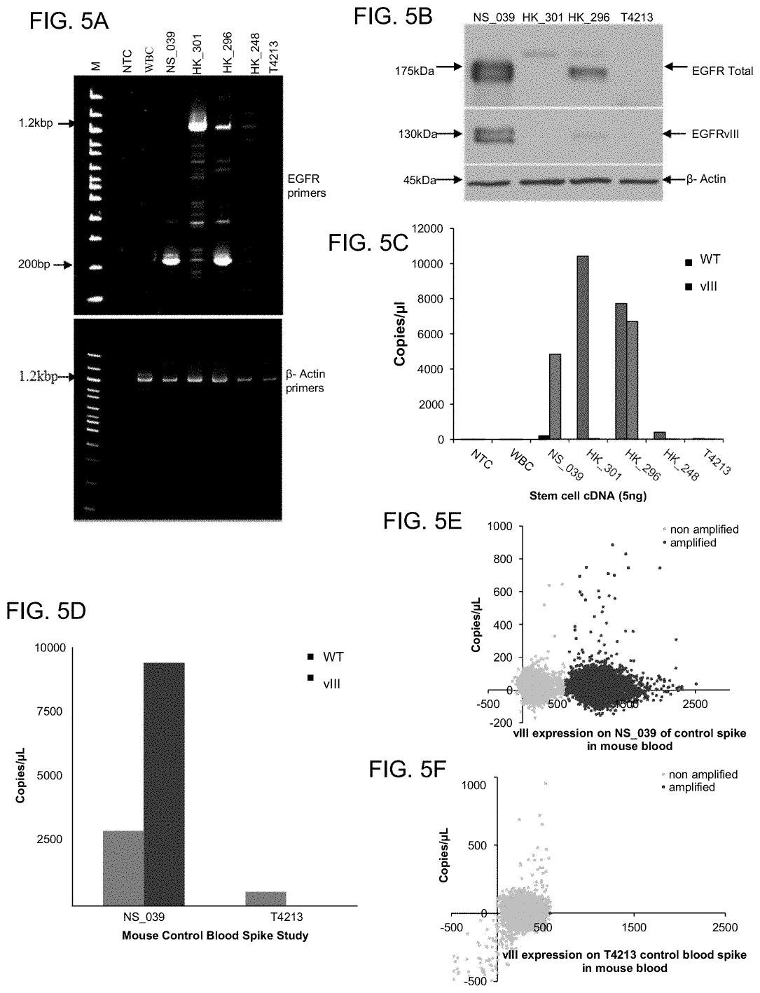

The presence or absence of EGFRvIII in patient-derived glioma stem cells (GSCs) (N5039, HK301, HK296, HK248 and T4213) was initially confirmed by conventional PCR using gene specific primers to EGFR (FIG. 5A). Higher vIII was observed in NS039 and HK296 cell lines. HK301 was also found to have a small amount of EGFRvIII, but a higher abundance of EGFR WT. HK248 and T4213 did not contain any EGFRvIII. The above findings were confirmed by Western Blot (FIG. 5B). Following validation by conventional PCR and Western Blot, our dPCR assay was run on cDNA extracted from our patient-derived GSCs. HK296 was found to have the highest copies per microliter of vIII followed by NS039 (FIG. 5C). HK301 was also found to contain very low copies of vIII (43 copies/μL), but this is obscured by its abundance of EGFR wildtype (FIG. 5C). HK296 was also found to have high copy numbers of both the wildtype and vIII isoforms together. HK248 and T4213 were negative for both EGFR WT and vIII (FIG. 5C). Given these results, we conclude the dPCR assay is able to quantitatively detect the EGFRvIII mutation, as well as the wildtype isoform.

Next, we sought to determine the ability of our assay to detect EGFRvIII in the presence of other cell populations. One thousand NS039 and T4213 cells were spiked into mouse whole blood, and then processed on CelSee micro-fluidic chips, as described in the methods section. Cells isolated from CelSee chips underwent further processing to extract cDNA. One nanogram of cDNA was analyzed using our dPCR assay. EGFRvIII was detected in abundance in NS039 recovered from spiked blood (FIG. 5D and FIG. 5E), but was not detected in T4213 (FIG. 5D and FIG. 5F). This suggests that our assay is not influenced by other cell types and background genomic material.

### Orthotopic GBM Mouse Xenografts

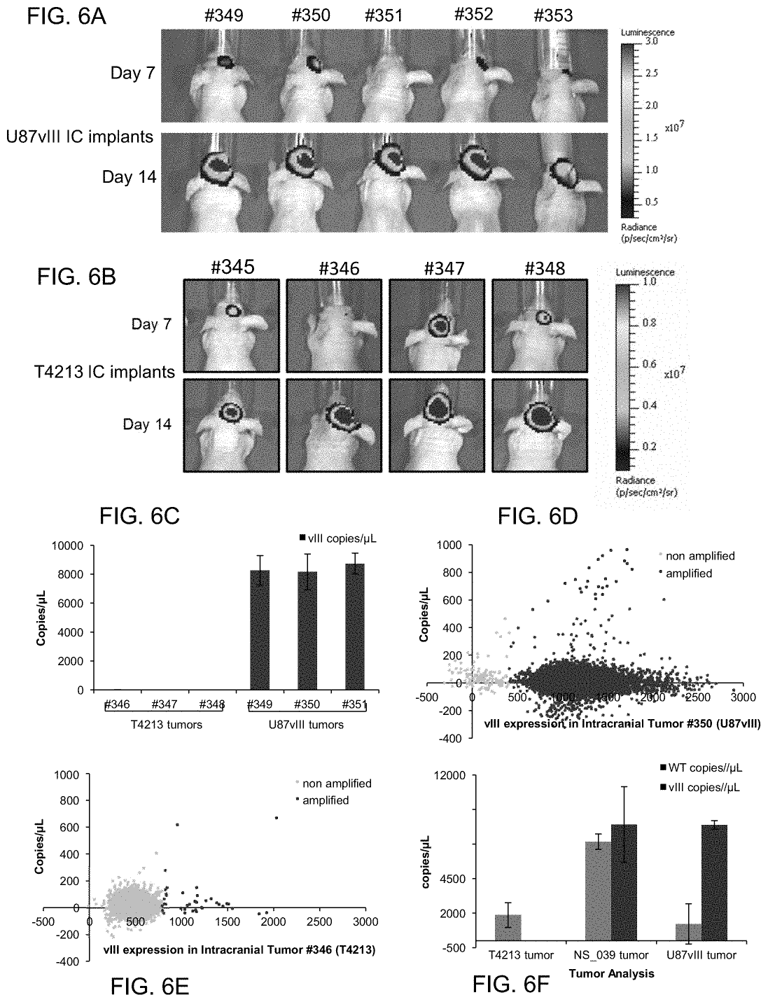

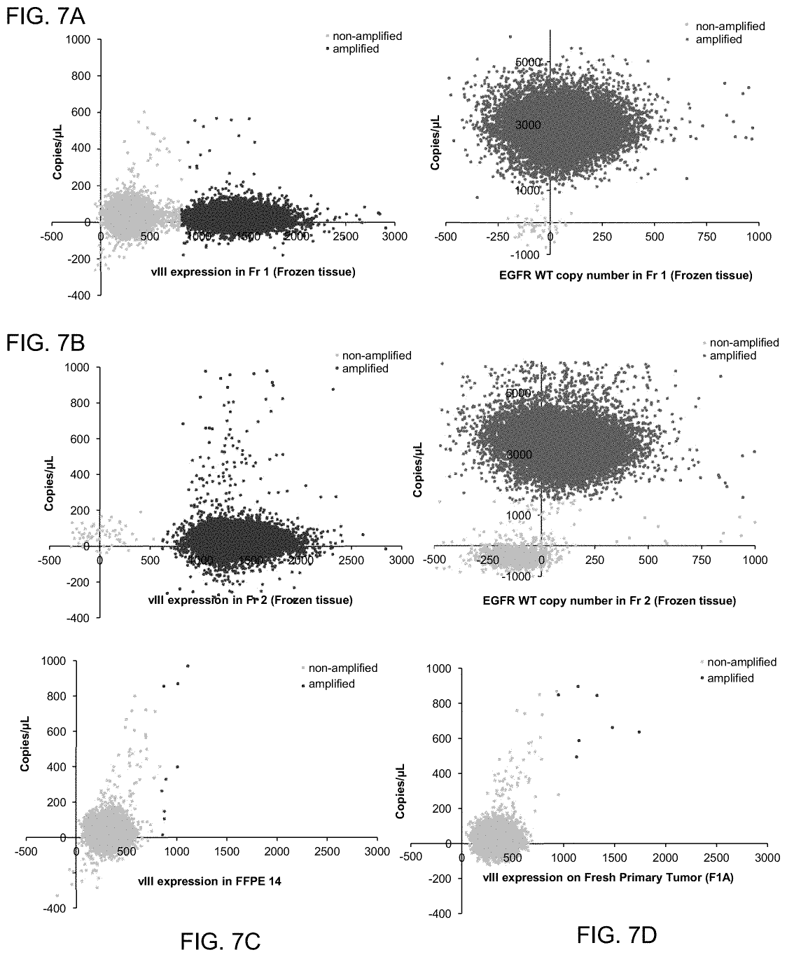

We validated our assay using both flank and orthotopic xenografts of glioma cells. For intracranial tumor implants, growth was monitored weekly by BLI (FIG. 6A and FIG. 6B), and mice were humanely euthanized four to six weeks post implantation or when neurological defects became significant. Immediately following euthanasia, tumor tissue was dissected out of brains for histological and molecular analysis by dPCR. Six tumors were analyzed using our dPCR assay: animals 346, 347 and 348 were inoculated with T4213 and animals 349, 350 and 351 were inoculated with U87vIII cells. EGFRvIII was not detected in tumors from animals 346, 347, and 348, while tumors from animals 349, 350, and 351 demonstrated an abundance of vIII (FIG. 6C). Isolated scatter plots confirm that tumors from mice inoculated with U87 vIII have strong VIC signal indicating that they are positive for vIII, whereas tumors from mice inoculated T4213 had no appreciable VIC signal (FIG. 6D and FIG. 6E). Direct comparison of different xenografts highlights the ability of our assay to identify differences in vIII from distinct tumor types (FIG. 6F). The vIII variant was found in both NS039 flank tumors as well as U87 vIII intracranial tumors, but was absent T4213 intracranial tumors (FIG. 6F). Analysis of NS039 flank tumors also revealed increased copy numbers of both EGFR WT and EGFRvIII demonstrating that our assay has the potential to identify the genotype of single cells from three-dimensional tumors (FIG. 6F)

cfRNA Quantification in Patient Derived GSCs Growth Media

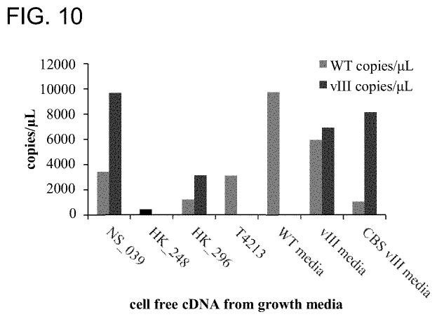

In order to detect the EGFRvIII mutation in cell free RNA (cfRNA) we collected growth media from different patient-derived GSC and human derived glioma cell lines. FIG. 10 represents vIII expression in cfRNA extracted from growth media of different patient-derived GSCs and human derived glioma cell lines. cfRNA is extracted from 200 μL of growth media as described in method section and spiked into whole mouse blood. Our dPCR assay was then run on cDNA extracted from total cfRNA. The expression pattern of vIII in cfRNA resembles vIII expression in actual GSC cell lines.

### Patient Samples Analyses

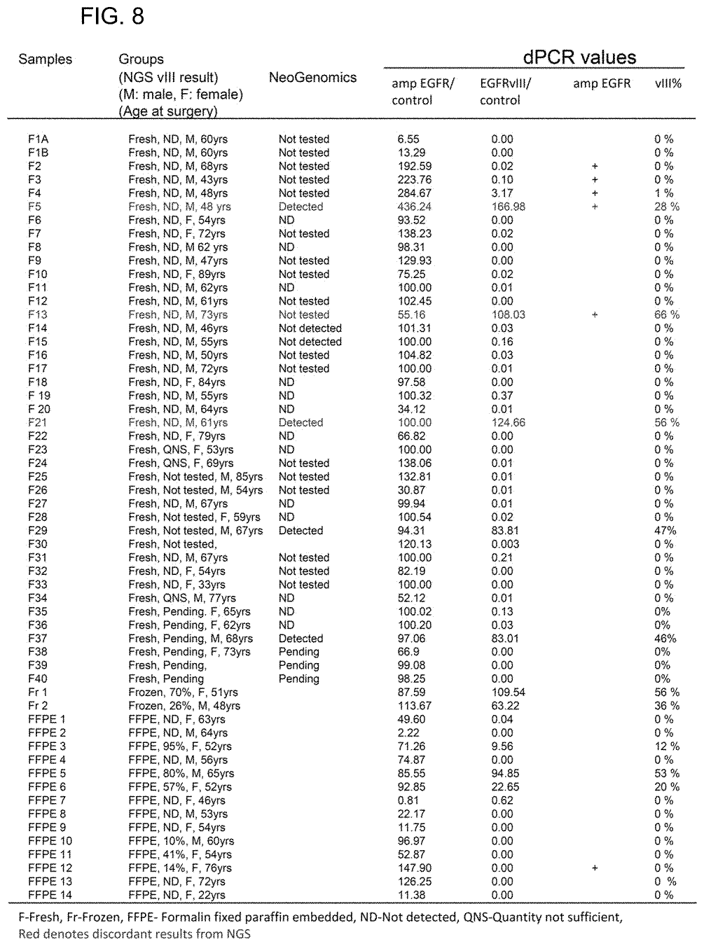

We utilized our dPCR assay to analyze patient-derived tumor specimens in a blinded pilot study approved by the Institutional Review Board at the University of Pennsylvania. Our dPCR assay was used to analyze cDNA derived from close to forty fresh frozen tissues, fourteen FFPE blocks, and two frozen tissue specimens (FIG. 8). EGFR WT was considered positive if the copies per microliter of amplified EGFR were greater or equal to one and half times the copies per microliter of control or reference allele. The percentage of EGFRvIII was determined by finding the ratio of EGFRvIII to total EGFR (EGFR WT and EGFRvIII),

$$\frac{vIII copies \text{/} µL}{vIII copies \text{/} µL + WTcopies \text{/} µL} \times 100.$$

In accordance with NGS conventions, EGFRvIII was considered positive if the percentage of vIII was greater than ten percent. Patients were not enrolled in clinical trials if the percentage of EGFRvIII was less than ten percent as determined by NGS.

RNA was extracted from FFPE samples that had been fixed and embedded approximately two to five years ago. Our assay was able to detect the vIII variant in tissue specimens known to carry vIII, but did not identify the mutation in tissue blocks that had been classified as negative (FIG. 8). Fourteen FFPE tissue blocks were analyzed for vIII via dPCR. We were able to detect vIII in three out of six blocks that had been classified as vIII positive by NGS and IHC, Interestingly, those three specimens were found to have higher expressions of vIII as determined by IHC (200, 140, and 200 fold respectively) when compared to NGS percentages of 80%, 57%, and 95% respectively. RNA extracted from FFPE samples undergo fragmentation over time, which explains detection of vIII by dPCR in higher vIII % samples.

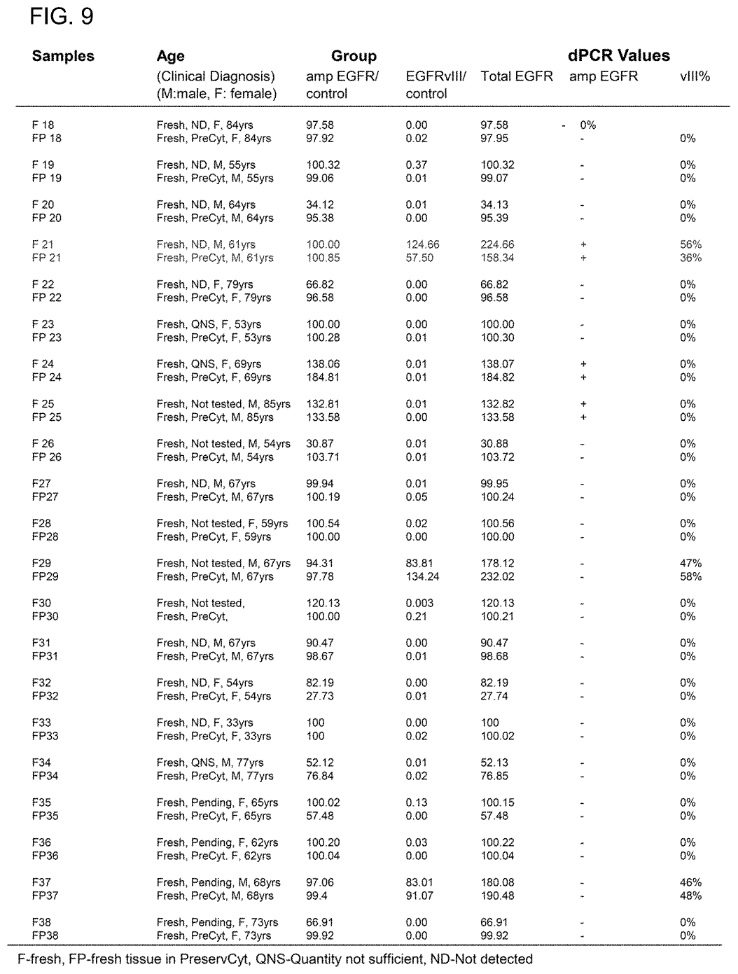

Fresh primary tumor samples were tested in parallel for the presence of EGFRvIII with both our dPCR assay as well as NGS. Our assay was able to duplicate the findings of NGS for the majority of the specimens. However, we encountered three discordant results (samples F5, F13 and F21 in FIG. 8), where the tumor specimen that had been characterized as vIII negative by NGS, but was found to be vIII positive by our dPCR assay. To investigate this further, the corresponding FFPE tissue block for the F5 sample was re-sequenced and also examined by RT-PCR. Results of the repeat NGS sequencing and the RT-PCR profiling confirmed that F5 sample is indeed EGFRvIII positive. Similarly, vIII expression in sample F21 was also confirmed by RT-PCR. Expression of vIII in all of these samples (F5, F13 and F21) was also confirmed by semi-quantitative PCR. This suggests that the dPCR assay we have developed is able to detect the EGFRvIII variant with a high degree of accuracy, and may be able identify the variant in specimens that were initially misclassified as negative for vIII. Furthermore, we were able to detect vIII in fresh tumor specimens down to as little as one hundred μg. Storage of tumor specimens in a preservative such as PreservCyt® did not affect the quality or readout of our assay (FIG. 9). The turnaround time for the dPCR assay is less than twenty-four hours, allowing for rapid detection of the EGFRvIII variant and thus far earlier enrollment of patients on trials for vIII-targeted therapies.

### Discussion

Glioblastoma is the most common and clinically aggressive form of primary brain tumor. Mutations in EGFR are a common finding. The variant III version of EGFR is found in up to thirty percent of GBM patients, and is produced by an in-frame deletion of 801 bp from exon 2 to exon 7 (EGFRvIII)^{2,3}. Loss of the EGFR extracellular domain in EGFRvIII is associated with ligand-independent constitutive activation, and elevated activation of signaling pathways implicated in proliferation, motility, and metastatic dissemination.^{21,22} Antibodies against EGFR and small molecules targeting EGFR have been used to treat patients with GBM, but were not clinically efficacious.²³ CAR-T cells targeting EGFRvIII can selectively eliminate EGFRvIII tumor cells.²³⁻²⁵ Although CAR-T efficiently eliminates EGFRvIII tumor cells in some patients, it did not produce a survival advantage in recurrent disease.¹³ Early targeting of GBM cells harboring the EGFRvIII mutation may circumvent issues involved in recurrence. Targeting the vIII mutant oncoprotein with a peptide vaccine strategy (rindopepimut) is another approach. In phase 2 studies, rindopepimut was well-tolerated and produced an immune response.¹⁰ However antigen escape variants were noted, indicating that tumor cells suppress mutant EGFR expression in order to reach an optimal equilibrium for growth. Reemergence of the clonal EGFR mutation is observed after drug withdrawal, suggesting a highly specific, dynamic, and adaptive route by which cancers can evade therapies that target oncogenes maintained on extra chromosomal DNA²⁶.

Efforts have been made to validate biomarkers that reflect the genetic profile of GBM, which would facilitate outcome prediction as well as help design targeted therapies.^{24,25} It is also conceivable that the molecular characterization of tumor tissue, circulating tumor cells, microvesicles, and cfRNA would help to elucidate genomic variations that occur during tumor evolution and disease recurrence, including the accumulation of late chromosomal deletions, amplifications, and mutations. Presently, NGS is utilized to identify EGFR mutations from patient-derived tissue specimens. However, NGS can take up to three weeks to identify a mutation due to clinical workflows. Studies have attempted to map EGFRvIII mutation via qPCR and southern blot using series of primers corresponding to introns 1 to 6, and exon 7 and exon 8; however this process is time intensive.^{15,26,27} Furthermore, qPCR relies on reference controls, which may be problematic since expression levels of endogenous controls and their transcripts differ between samples. Also, qPCR does not offer adequate sensitivity for the detection of a low copy number mutation.

The digital PCR method provided herein allows for the direct quantification of nucleic acids by partitioning the reaction mixture into twenty-thousand wells. A positive well indicates the presence of a single molecule in a given reaction and produces a high-confidence measurement of the original target concentration. The digital PCR method described herein is able to quantify the presence of EGFRvIII at the level of one picogram in a background of six thousand picograms of EGFR wildtype (FIG. 2F).

We have utilized this assay to detect EGFRvIII in a small cohort of GBM tumor samples. We detected the presence of EGFRvIII in five out of thirty-eight total patients. Original FFPE tissue blocks were sent for clinical next generation sequencing at our institution, as well as in some cases to NeoGenomics Laboratories, Inc. Parallel testing was able to confirm the presence of EGFRvIII in the primary tumor, thus highlighting the sensitivity of our assay for detecting EGFRvIII in patient-derived tissue specimens. Our assay is able to rapidly and accurately characterize the EGFRvIII variant in brain tumors and other solid tumors when NGS is unable to detect the presence of the mutation.

This study focuses on the development of a very sensitive method for identifying EGFRvIII and amplified EGFR in tumors by dPCR. Our assay utilizes the unique sequence generated due to the fusion of Exon 2 and Exon 8. The dPCR assay for EGFRvIII is able to identify EGFRvIII mutation in cell lines (FIG. 4A-FIG. 4D), patient-derived stem cells (FIG. 5A-FIG. 5F), and in xenograft tumors (FIG. 6A-FIG. 6F). With this dPCR assay, EGFRvIII can be detected in patients within a day of resection (FIG. 1).

The results described in this report suggest that a dPCR strategy can be successful in detecting EGFRvIII deletion without the need to sequence the whole genome of a patient.

Inclusion of a digital PCR assay to detect the presence of EGFRvIII mutation following tumor resection will help guide the appropriate treatment. Early detection of EGFRvIII may allow for institution of targeted treatments, such as CAR-T, allowing for earlier elimination of aggressive tumor populations. Detection of the EGFRVIII mutation via digital PCR represents a superior diagnostic assay in terms of speed and sensitivity.

### Example 3

### Improved Detection of A289V and R108K EGFR Variants

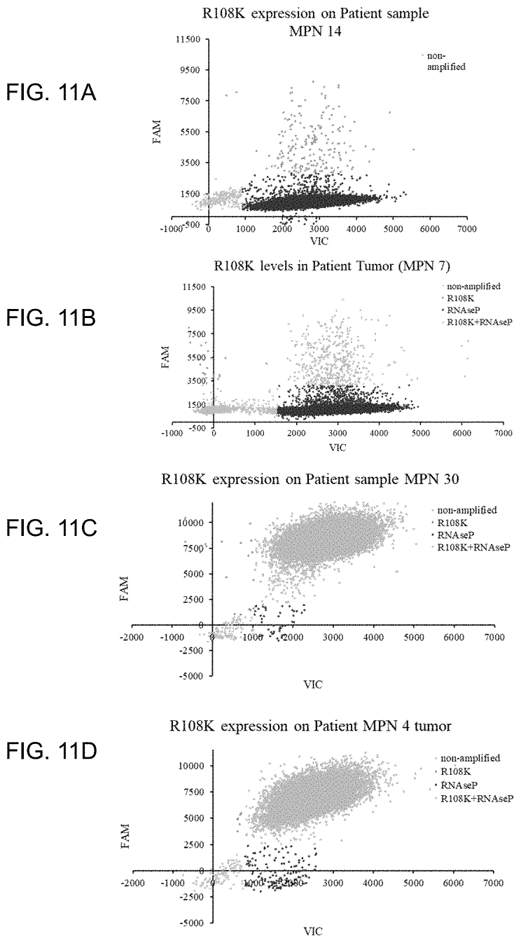

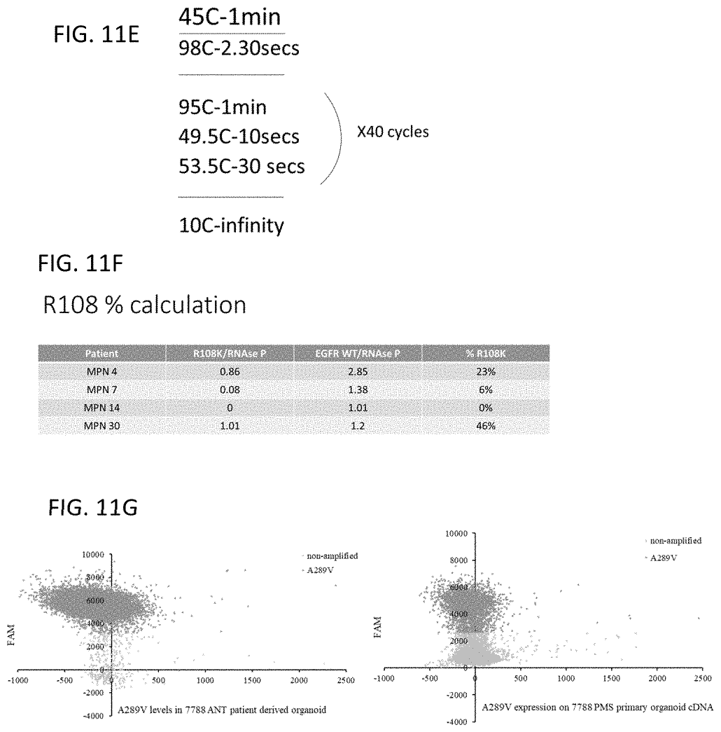

The detection of EGFR missense mutations, including A289V and R108K, has been improved using the dPCR conditions provided in FIG. 11E. Samples were obtained and dPCR assays were performed using a workflow such as that provided in FIG. 1 and the methods described in Examples 1 and 2. The R108K mutation was detected in patient tumor samples (FIG. 11A-11D) and the A289V mutation was detected in patient-derived organoid samples (FIG. 11G).

### REFERENCES

1. Li G, Wong A J. EGF receptor variant III as a target antigen for tumor immunotherapy. Expert review of vaccines. 2008; 7(7):977-985.
2. Brennan C W, Verhaak R G, McKenna A, et al. The somatic genomic landscape of glioblastoma. Cell. 2013; 155(2):462-477.
3. Hatanpaa K J, Burma S, Zhao D, Habib A A. Epidermal growth factor receptor in glioma: signal transduction, neuropathology, imaging, and radioresistance. Neoplasia (New York, N.Y.). 2010; 12(9):675-684.
4. Guo G, Gong K, Wohlfeld B, Hatanpaa K J, Zhao D, Habib A A. Ligand-Independent EGFR Signaling. Cancer research. 2015; 75(17):3436-3441.
5. Choi B D, Archer G E, Mitchell D A, et al. EGFRvIII-targeted vaccination therapy of malignant glioma. Brain pathology (Zurich, Switzerland). 2009; 19(4):713-723.
6. Huang P H, Xu A M, White F M. Oncogenic EGFR signaling networks in glioma. Science signaling. 2009; 2(87):re6.
7. Stupp R, Mason W P, van den Bent M J, et al. Radiotherapy plus concomitant and adjuvant temozolomide for glioblastoma. The New England journal of medicine. 2005; 352(10):987-996.
8. Padfield E, Ellis H P, Kurian K M. Current Therapeutic Advances Targeting EGFR and EGFRvIII in Glioblastoma. Frontiers in oncology. 2015; 5:5.
9. Karpel-Massler G, Schmidt U, Unterberg A, Halatsch ME. Therapeutic inhibition of the epidermal growth factor receptor in high-grade gliomas: where do we stand? Molecular cancer research: MCR. 2009; 7(7):1000-1012.
10. Schuster J, Lai R K, Recht L D, et al. A phase II, multicenter trial of rindopepimut (CDX-110) in newly diagnosed glioblastoma: the ACT III study. Neuro-oncology. 2015; 17(6):854-861.
11. Sampson J H, Heimberger A B, Archer G E, et al. Immunologic escape after prolonged progression-free survival with epidermal growth factor receptor variant III peptide vaccination in patients with newly diagnosed glioblastoma. Journal of clinical oncology: official journal of the American Society of Clinical Oncology. 2010; 28(31):4722-4729.
12. Newick K, O'Brien S, Moon E, Albelda S M. CAR T Cell Therapy for Solid Tumors.

Annual review of medicine. 2017; 68:139-152.
13. O'Rourke D M, Nasrallah M P, Desai A, et al. A single dose of peripherally infused

EGFRvIII-directed CAR T cells mediates antigen loss and induces adaptive resistance in patients with recurrent glioblastoma. Science translational medicine. 2017; 9(399).
14. Gupta R G, Somer R A. Intratumor Heterogeneity: Novel Approaches for Resolving Genomic Architecture and Clonal Evolution. Molecular cancer research: MCR. 2017; 15(9):1127-1137.
15. Yoshimoto K, Dang J, Zhu S, et al. Development of a real-time RT-PCR assay for detecting EGFRvIII in glioblastoma samples. Clinical cancer research: an official journal of the American Association for Cancer Research. 2008; 14(2):488-493.
16. Alikian M, Ellery P, Forbes M, et al. Next-Generation Sequencing-Assisted DNA-Based Digital PCR for a Personalized Approach to the Detection and Quantification of Residual Disease in Chronic Myeloid Leukemia Patients. The Journal of molecular diagnostics: JMD. 2016; 18(2):176-189.
17. Kudo L C, Vi N, Ma Z, et al. Novel Cell and Tissue Acquisition System (CTAS): microdissection of live and frozen brain tissues. PLoS One. 2012; 7(7):e41564.
18. Macarthur K M, Kao G D, Chandrasekaran S, et al. Detection of brain tumor cells in the peripheral blood by a telomerase promoter-based assay. Cancer Res. 2014; 74(8):2152-2159.
19. Picelli S, Faridani O R, Bjorklund A K, Winberg G, Sagasser S, Sandberg R. Full-length RNA-seq from single cells using Smart-seq2. Nature protocols. 2014; 9(1):171-181.
20. Whale A S, Huggett J F, Cowen S, et al. Comparison of microfluidic digital PCR and conventional quantitative PCR for measuring copy number variation. Nucleic acids research. 2012; 40(11):e82.
21. Thorne A H, Zanca C, Furnari F. Epidermal growth factor receptor targeting and challenges in glioblastoma. Neuro Oncol. 2016.
22. Wieduwilt M J, Moasser M M. The epidermal growth factor receptor family: biology driving targeted therapeutics. Cellular and molecular life sciences: CMLS. 2008; 65(10):1566-1584.
23. Johnson L A, Sampson J H. Immunotherapy approaches for malignant glioma from 2007 to 2009. Current neurology and neuroscience reports. 2010; 10(4):259-266.
24. Morgan R A, Johnson L A, Davis J L, et al. Recognition of glioma stem cells by genetically modified T cells targeting EGFRvIII and development of adoptive cell therapy for glioma. Human gene therapy. 2012; 23(10):1043-1053.
25. Sampson J H, Choi B D, Sanchez-Perez L, et al. EGFRvIII mCAR-modified T-cell therapy cures mice with established intracerebral glioma and generates host immunity against tumor-antigen loss. Clinical cancer research: an official journal of the American Association for Cancer Research. 2014; 20(4):972-984.
26. Nathanson D A, Gini B, Mottahedeh J, et al. Targeted therapy resistance mediated by dynamic regulation of extrachromosomal mutant EGFR DNA. Science (New York, NY.). 2014; 343(6166):72-76.
27. Koga T, Li B, Figueroa J M, et al. Mapping of genomic EGFRvIII deletions in glioblastoma: insight into rearrangement mechanisms and biomarker development. Neuro-oncology. 2018; 20(10):1310-1320.
28. Bhat S, Herrmann J, Armishaw P, Corbisier P, Emslie K R. Single molecule detection in nanofluidic digital array enables accurate measurement of DNA copy number. Analytical and bioanalytical chemistry. 2009; 394(2):457-467.
29. Kreutz J E, Munson T, Huynh T, Shen F, Du W, Ismagilov R F. Theoretical design and analysis of multivolume digital assays with wide dynamic range validated experimentally with microfluidic digital PCR. Analytical chemistry. 2011; 83(21):8158-8168.
30. Pohl G, Shih Ie M. Principle and applications of digital PCR. Expert review of molecular diagnostics. 2004; 4(1):41-47.
31. Day E, Dear P H, McCaughan F. Digital PCR strategies in the development and analysis of molecular biomarkers for personalized medicine. Methods (San Diego, Calif). 2013; 59(1):101-107.
32. Vogelstein B, Kinzler K W. Digital PCR. Proceedings of the National Academy of Sciences of the United States of America. 1999; 96(16):9236-9241.

All publications cited in this specification are incorporated herein by reference in their entireties. U.S. Provisional Patent Application No. 62/891,765, filed Aug. 26, 2019, and its Sequence Listing are incorporated by reference. While the invention has been described with reference to particular embodiments, it will be appreciated that modifications can be made without departing from the spirit of the invention. Such modifications are intended to fall within the scope of the appended claims.

1. A method of detecting epidermal growth factor receptor - variant III (EGFRvIII), the method comprising generating a cDNA sample from RNA present in a biological sample, contacting the cDNA sample with a forward primer that binds EGFR exon 1, a reverse primer that binds EGFR exon 8, and a minor groove binder (MGB) probe comprising an oligonucleotide having at least the sequence of: 5′ AAA GGT AAT TAT 3′ (SEQ ID NO: 3) that binds the junction of EGFR exons 1 and 8, and performing digital PCR in order to detect EGFRvIII.

2. The method according to claim 1, wherein the forward primer comprises 5′ TCG GGC TCT GGA GGA AA 3′ (SEQ ID NO: 1) and/or the reverse primer comprises 5′ CTT CCT CCA TCT CAT AGC TGT C 3′ (SEQ ID NO: 2).

3. The method according to claim 1, wherein said EGFRvIII is detected with a limit of detection (LOD) of about 0.625×10⁻⁵ ng/ul and/or a limit of quantification (LOQ) of about 0.003%.

4. The method according to claim 1, further comprising amplifying the cDNA generated from the RNA present in the biological sample.

5. The method according to claim 1, wherein the biological sample is selected from whole blood, PBMC, serum, a fresh, frozen, or preserved tumor sample, a tissue sample, CSF, a lymphatic fluid sample, a cell line, urine, and circulating tumor cells.

6. The method according to claim 1, wherein the biological sample is tumor tissue.

7. The method according to claim 1, wherein the MGB probe is labeled with a dye selected from FAM, VIC, TET, and NED.

8. The method according to claim 1, wherein digital PCR is performed using one cycle of 96° C. for 10 minutes, followed by 45 cycles of 54° C. for 2 minutes and 98° C. for 30 seconds and a final extension at 54° C. for 2 minutes.

9. A method of detecting EGFR, the method comprising generating a cDNA sample from RNA present in a biological sample, further comprising contacting the cDNA sample with a forward primer that binds EGFR exon 5, a reverse primer that binds EGFR exon 6, and a MGB probe comprising an oligonucleotide having at least the sequence of: 5′ AGG AGA ACT GCC 3′ (SEQ ID NO: 6) that binds the junction of EGFR exons 5 and 6, and performing digital PCR in order to detect wildtype EGFR.

10. The method according to claim 9, wherein the forward primer that binds EGFR exon 5 comprises 5′ AAG TGT GAT CCA AGC TGT CC 3′ (SEQ ID NO: 4) and/or the reverse primer that binds EGFR exon 6 comprises 5′ TGC TGG GCA CAG ATG ATT T 3′(SEQ ID NO: 5).

11. The method according to claim 9, further comprising amplifying the cDNA generated from the RNA present in the biological sample.

12. The method according to claim 9, wherein the biological sample is selected from whole blood, PBMC, serum, a fresh, frozen, or preserved tumor sample, a tissue sample, CSF, a lymphatic fluid sample, a cell line, urine, and circulating tumor cells.

13. The method according to claim 9, wherein the biological sample is tumor tissue.

14. The method according to claim 9, wherein the MGB probe is labeled with a dye selected from FAM, VIC, TET, and NED.

15. The method according to claim 9, wherein digital PCR is performed using one cycle of 96° C. for 10 minutes, followed by 45 cycles of 54° C. for 2 minutes and 98° C. for 30 seconds and a final extension at 54° C. for 2 minutes.

16. A method for treating a cancer characterized by expression of epidermal growth factor receptor—variant III (EGFRvIII), the method comprising generating a cDNA sample from RNA present in the biological sample, contacting the cDNA sample with a forward primer that binds EGFR exon 1, a reverse primer that binds EGFR exon 8, and a MGB probe having an oligonucleotide comprising at least the sequence of: 5′ AAA GGT AAT TAT 3′ (SEQ ID NO: 3) that binds the junction of EGFR exon 1 and 8, and performing digital PCR in order to detect the EGFRvIII, wherein the presence of amplified sequences indicates a cancer associated with EGFRvIII, and treating the subject for the cancer.

17. The method according to claim 16, wherein the sample is a brain tumor sample and/or the cancer is glioblastoma.


SEQUENCE LISTING (15 sequences)

SEQ ID NO: 1 - DNA, 17 bp, Artificial Sequence
  primer
tcgggctctggaggaaa

SEQ ID NO: 2 - DNA, 22 bp, Artificial Sequence
  primer
cttcctccatctcatagctgtc

SEQ ID NO: 3 - DNA, 12 bp, Artificial Sequence
  primer
aaaggtaattat

SEQ ID NO: 4 - DNA, 20 bp, Artificial Sequence
  primer
aagtgtgatccaagctgtcc

SEQ ID NO: 5 - DNA, 19 bp, Artificial Sequence
  primer
tgctgggcacagatgattt

SEQ ID NO: 6 - DNA, 12 bp, Artificial Sequence
  primer
aggagaactgcc

SEQ ID NO: 7 - DNA, 57 bp, Artificial Sequence
  primer
  misc_feature (57)..(57): n is a, c, g, or t
aagcagtggtatcaacgcagagtacttttttttttttttttttttttttttttttvn

SEQ ID NO: 8 - DNA, 30 bp, Artificial Sequence
  primer
  modified_base (28)..(28): riboguanosine
  modified_base (29)..(29): riboguanosine
  modified_base (30)..(30): locked nucleic acid modified guanosine
aagcagtggtatcaacgcagagtacatggg

SEQ ID NO: 9 - DNA, 23 bp, Artificial Sequence
  primer
aagcagtggtatcaacgcagagt

SEQ ID NO: 10 - DNA, 18 bp, Artificial Sequence
  primer
atgcgaccctccgggacg

SEQ ID NO: 11 - DNA, 22 bp, Artificial Sequence
  primer
attccgttacacactttgcggc

SEQ ID NO: 12 - DNA, 98 bp, Homo sapiens
gcgctgctggctgcgctctgcccggcgagtcgggctctggaggaaaagaagtttgccaaggcacgagtaacaagctcacgcagttgggcacttttgaa

SEQ ID NO: 13 - DNA, 109 bp, Homo sapiens
atgcgaccctccgggacggccggggcagcgctcctggcgctgctggctgcgctctgcccggcgagtcgggctctggaggaaaagaaaggtaattatgtggtgacagatc

SEQ ID NO: 14 - DNA, 3633 bp, Homo sapiens
  CDS (1)..(3633)
atgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgactgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatga

SEQ ID NO: 15 - protein, 1210 aa, Homo sapiens [sequence omitted]